SEQUENCE LISTING (2 sequences)

SEQ ID NO: 1 - RNA, 31 nt, Artificial Sequence
  Synthetic
  misc_feature (1)..(1): 2′O-methyl Guanosine
  misc_feature (2)..(2): 2′O-methyl Uridine
  misc_feature (3)..(4): 2′O-methyl Guanosine
  misc_feature (5)..(5): 2′O-methyl Adenosine
  misc_feature (6)..(6): 2′Fluoro Cytosine
  misc_feature (7)..(7): 2′Fluoro Uridine
  misc_feature (8)..(8): 2′O-methyl Adenosine
  misc_feature (9)..(9): 2′Fluoro Uridine
  misc_feature (10)..(10): 2′O-methyl Adenosine
  misc_feature (11)..(12): 2′Fluoro Cytosine
  misc_feature (13)..(13): 2′O-methyl Guanosine
  misc_feature (14)..(14): 2′Fluoro Cytosine
  misc_feature (15)..(15): 2′O-methyl Guanosine
  misc_feature (16)..(16): 2′Fluoro Uridine
  misc_feature (17)..(18): 2′O-methyl Adenosine
  misc_feature (19)..(19): 2′Fluoro Uridine
  misc_feature (20)..(20): 2′O-methyl Guanosine
  misc_feature (21)..(21): 2′Fluoro Cytosine
  misc_feature (22)..(22): 2′O-methyl Uridine
  misc_feature (23)..(23): 2′Hydroxy Guanosine
  misc_feature (24)..(24): 2′O-methyl Cytosine
  misc_feature (25)..(25): 2′Fluoro Cytosine
  misc_feature (26)..(26): 2′Fluoro Uridine
  misc_feature (27)..(28): 2′O-methyl Cytosine
  misc_feature (29)..(29): 2′O-methyl Adenosine
  misc_feature (30)..(30): 2′O-methyl Cytosine
  misc_feature (31)..(31): inverted deoxy thymidine
nnnnnnnnnnnnnnnnnnnnnnnnnnnnnnn

SEQ ID NO: 2 - RNA, 15 nt, Artificial Sequence
  Synthetic
  misc_feature (1)..(1): 2′O-methyl Guanosine
  misc_feature (2)..(2): 2′O-methyl Guanosine
  misc_feature (3)..(3): 2′O-methyl Cytosine
  misc_feature (4)..(5): 2′O-methyl Guanosine
  misc_feature (6)..(6): 2′O-methyl Uridine
  misc_feature (7)..(7): 2′O-methyl Adenosine
  misc_feature (8)..(8): 2′O-methyl Uridine
  misc_feature (9)..(9): 2′O-methyl Adenosine
  misc_feature (10)..(10): 2′O-methyl Guanosine
  misc_feature (11)..(11): 2′O-methyl Uridine
  misc_feature (12)..(13): 2′O-methyl Cytosine
  misc_feature (14)..(14): 2′O-methyl Adenosine
  misc_feature (15)..(15): 2′O-methyl Cytosine
nnnnnnnnnnnnnnn

(19) United States
(12) Patent Application Publication
(10) Pub. No.: US 2024/0226307 A9
(43) Pub. Date: Jul. 11, 2024

(54) NON-IMMUNOGENIC POEGMA-APTAMER CONJUGATES

(71) Applicant: Duke University, Durham, NC (US)

(72) Inventors: Ashutosh Chilkoti, Durham, NC (US); Bruce Sullenger, Durham, NC (US); Imran Ozer, Durham, NC (US); Angus Hucknall, Durham, NC (US); Juliana Layzer, Durham, NC (US); George Pitoc, Durham, NC (US)

(21) Appl. No.: 18/277,915

(22) PCT Filed: Feb. 22, 2022
(86) PCT No.: PCT/US2022/017349

§ 371 Date: Aug. 18, 2023

Related U.S. Application Data
(60) Provisional application No. 63/151,878, filed on Feb. 22, 2021.
(60) Provisional application No. 63/274,839, filed on Nov. 2, 2021.

(51) Int. Cl. A61K 47/60; A61K 31/7088; A61P 7/02; C12N 15/115

(52) U.S. Cl. CPC A61K 47/60; A61K 31/7088; A61P 7/02; C12N 15/115

(57) ABSTRACT

Disclosed are POEGMA-aptamer conjugates with a reduced or eliminated host-immune response. An example conjugate includes an aptamer conjugated to a POEGMA having a plurality of side chains, where each side chain includes 1 to 6 monomers of ethylene glycol repeated in tandem. Also disclosed are methods of making the conjugate and methods of using the conjugate. An example method of use includes a method of controlling coagulation in a subject.

### CROSS-REFERENCE TO RELATED APPLICATIONS

This application claims the benefit of and priority to U.S. Provisional Application No. 63/151,878, filed on Feb. 22, 2021; and U.S. Provisional Application No. 63/274,839, filed on Nov. 2, 2021, the entire contents of each which are hereby incorporated by reference.

### STATEMENT REGARDING FEDERALLY SPONSORED RESEARCH

This invention was made with government support under grant number R41HL139234-0l awarded by the National Institutes of Health. The government has certain rights in the invention.

### SEQUENCE LISTING

The sequence listing is filed with the application and is incorporated by reference herein. The sequence listing text file “028193-9375-WO01_As_Filed_Sequence_Listing.txt” was created on Feb. 22, 2022, and is 41606862 bytes in size.

### BACKGROUND

Ribonucleic acid (RNA) aptamers and conjugates thereof are an emerging class of drugs. Unfortunately, RNA aptamers have significant delivery challenges. One method that can be used to alleviate some of these delivery challenges is to covalently attach polyethylene glycol (PEG) to aptamers—termed PEGylation. However, PEG induces varying levels of PEG antibodies upon treatment and activates the complement system, which can abrogate their clinical use and trigger severe infusion reactions to PEGylated drugs in individuals with a high titer of PEG antibodies. Perhaps an even more significant limitation of PEG is the prevalence of varying titers of pre-existing PEG antibodies in up to 70% of humans who have never been treated with PEGylated therapeutics, likely because of PEG's ubiquitous use as an excipient in drugs, laxatives, and in consumer products.

Complicating the delivery of RNA aptamers is the issue of PEG-RNA conjugates initiating a significantly different antigen response compared to a PEG-protein conjugate. For example, PEG-RNA conjugates initiate a thymus-independent antigen response, compared to a thymus-dependent antigen response seen with PEG-protein conjugates. See Chen et al., ACS Nano, 15, 14022-14048, 2021. This makes it difficult to predict how the immune system will respond to non-protein conjugates (e.g., RNA aptamer conjugates) when attempting to replace PEG.

### SUMMARY

In one aspect, provided are conjugates including an aptamer; and a poly[oligo(ethylene glycol) methyl ether methacrylate] (POEGMA) conjugated to the aptamer, wherein the POEGMA has a poly(methyl methacrylate) backbone and a plurality of side chains covalently attached to the backbone, each side chain comprising 1 to 6 monomers of ethylene glycol (EG) repeated in tandem, wherein the conjugate does not induce an anti-POEGMA antibody response.

In another aspect, provided are conjugates including an aptamer that includes SEQ ID NO: 1; and a POEGMA conjugated to the aptamer, wherein the POEGMA has a poly(methyl methacrylate) backbone and a plurality of side chains covalently attached to the backbone, each side chain comprising 1 to 6 monomers of EG repeated in tandem, wherein the conjugate does not induce an anti-POEGMA antibody response, and wherein the conjugate is not reactive with pre-existing anti-PEG antibodies in a subject.

In another aspect, provided are methods of making a polymer-aptamer conjugate, the method including conjugating a POEGMA having a poly(methyl methacrylate) backbone and a plurality of side chains covalently attached to the backbone, each side chain comprising 1 to 6 monomers of EG repeated in tandem to a 5′ end of an aptamer to provide a conjugate, wherein the conjugate does not induce an anti-POEGMA antibody response.

In another aspect, provided are methods of controlling coagulation in a subject, the method comprising administering to a subject in need thereof a therapeutically effective amount of the disclosed conjugate, wherein administering the conjugate prevents or reduces blood clot formation in the subject.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIG. 1 is a schematic showing PEGylated RNA apatamers v. POEGMA conjugated RNA apatamers.

FIG. 2 shows synthesis of RB005 conjugates. (A) Schematic of POEGMA-RB005 conjugate. RB005 structure has chemical modifications on the guanidine (G), cytosine (C), adenine (A), and uracil (U) bases. (B) SEC traces of RB005 variants. RB005 and DBCO-RB005 contained ˜2 mass % of the dimer, as indicated by the small peak eluting at ˜20 min, as measured by SEC-MALS.

FIG. 3 shows POEGMA-RB005 anticoagulant activity in vitro. (A) POEGMA-RB005's mechanism of action. (B) Anticoagulant activity of the conjugates was tested in an activated partial thromboplastin time (aPTT) assay. Five independent assays were performed in duplicate. Data are presented as the mean aPTT clotting time in seconds±standard [deviation] and are plotted as a function of aptamer plasma concentration. Data were analyzed using an unpaired t-test.

FIG. 4 shows POEGMA-RB005 in vivo antithrombotic efficacy. (A) Experimental scheme. (B) In vivo antithrombotic activity of POEGMA-RB005 and RB006 at equivalent i.v. injection dose of 2 mg kg⁻¹ body weight (n=3). The experiment was terminated after monitoring blood flow for 60 min. An equivalent injection volume of platelet-binding buffer (PBB) was used as a negative control (n=3). The blood flow at any given time was normalized to the blood flow pre-patch placement. Data were analyzed by two-way ANOVA, followed by post hoc Dunnett's multiple comparison test. Data are presented as the mean±standard error of the mean and were considered statistically significant when p<0.05.

FIG. 5 shows POEGMA-RB005 having no reactivity to induced PEG antibodies. (A) Indirect ELISA and (B) competitive ELISA were performed using an induced PEG antibody-positive murine plasma sample. In indirect ELISA, the antigens (n=4) were coated onto the surface and incubated with the plasma sample. OVA-PEG was used as a PEGylated positive control while PBS (diluent) was used as a negative control. In competitive ELISA, a dilution series of RB006, and RB005-POEGMA (n=4) or RB005 (PEG-minus negative control) were added to a murine plasma sample that contains PEG antibodies. This mixture was incubated on exendin-PEG conjugate coated microwell-plates. The decrease in absorbance only for RB006 indicates that the PEG in the RB006 in solution successfully competes with the exendin-PEG conjugate adsorbed on the surface from binding to the PEG antibodies in the murine plasma sample. Data were analyzed by two-way repeated-measures ANOVA followed by post-hoc Tukey's multiple comparison test. Data represented the mean±standard deviation of the mean and were considered statistically significant when p<0.05. Not significant (ns). * p<0.05, **p<0.01, *** p<0.001, and ****p<0.0001.

FIG. 6 shows POEGMA-RB005 does not induce anti-POEGMA antibodies, while RB006 treatment induces significant PEG-specific immune response. (A) Dosing and blood collection regimen. IgM response on (B) Day −7, (C) Day 14, (D) Day 35, and (E) Day 56. X-axis shows serum samples that were collected from mice (n=10) repeatedly treated with PBS, RB006, or POEGMA-RB005. Assay diluent is 0.2% (w/v) I-Block protein-based blocking reagent (Thermo Scientific) in PBS and used as a negative control. The ADA response (n=6) was measured for each mouse using a multiplexed Luminex immunoassay. OVA-PEG- and OVA-POEGMA-coupled beads were used to determine ADAs induced towards PEG or POEGMA, respectively. The OVA-coupled bead was used as a control for cross-reactivity to OVA. Data represented the mean ADA response in a treatment group and standard error of the mean. Data were analyzed by two-way repeated-measures ANOVA followed by post-hoc Tukey's multiple comparison test. Data were considered statistically significant when p<0.05. Not significant (ns). *p<0.05, **p<0.01, ***p<0.001, and ****p<0.0001.

FIG. 7 shows nuclear magnetic resonance spectrum of azide-functional POEGMA used in the synthesis of POEGMA-RB005. A 400 MHz Varian Inova spectrometer was used to acquire the data. Deuterated chloroform and trimethylsilane (TMS) were used as a solvent and reference, respectively. 64 repeats and 2 replicates were performed. Data were analyzed using ACD software (ACD Labs). Integral values correspond to the average number of hydrogens (H) present in the OEG side-chain (b; 4.4-3.4 ppm; 10H), chain end-group (c; 3.5-3.3 ppm; 3H), methylene protons (a; 4.4-4.0 ppm; 2H), backbone (d; 2.2-1.6 ppm; 2H), and backbone methacrylate (e; 1.1-0.7 ppm; 3H).

FIG. 8 shows tris-borate-EDTA (TBE)-urea polyacrylamide gel electrophoresis analysis of RB006 purification via anion-exchange chromatography. Lane A: RB005. Lane PEG: PEG does not run on the gel due to its neutral charge. Lane R: RB006 conjugation reaction, containing both unreacted RB005 and RB006; Lane FT: Flow through collected during column wash with low-salt buffer. Lane FX: Fraction #collected during elution with high salt buffer. Purified conjugate is collected in F2, and no signal corresponding to RB005 was detected in this fraction. F4 and F5 contained unreacted RB005.

FIG. 9 shows characterization of POEGMA and PEG conjugates of RB005. (A) SEC chromatogram of RB005, PEG, and RB006 and (B) DBCO-RB005, POEGMA, and POEGMA-RB005.

FIG. 10 shows hydrodynamic radius (R %) of RB005 variants. Dynamic light scattering (DLS) analysis of (A) RB006, (B) POEGMA-RB005. DLS was performed on a temperature-controlled DynaPro Plate Reader (Wyatt Technologies). Ten repeats were performed for each of the three replicates.

FIG. 11 shows POEGMA-RB005 having reversible anticoagulant activity comparable to RB006. (A) Watson-Crick base pairing mediated activity reversal using a complementary nucleic acid chain (RB007) as an antidote. (B) Dose-dependent reversible anticoagulant activity. One assay was performed in duplicate. Data were analyzed using two-way ANOVA, followed by post-hoc Tukey's multiple comparison test. Data represent the mean±standard error of the mean (SEM). A statistical test was considered significant when the p<0.05 **p<0.01; **** p<0.0001). Not significant (ns).

FIG. 12 shows dose-escalation of POEGMA-RB005. Dose-dependent in vivo antithrombotic activity of POEGMA-RB005 using PBB as a negative control (n=3-4 mice). The blood flow at any given time was normalized to pre-drug administration. Data show the mean+standard error of the mean. Data were analyzed using two-way ANOVA, followed by post-hoc Dunnett's multiple comparison test. Data showed the mean+standard error of the mean and were considered statistically significant when p<0.05. Not significant (ns). * p<0.05, ****p<0.0001.

FIG. 13 shows RB007 antidote oligonucleotide reverses anticoagulant POEGMA-RB005 activity in a murine saphenous vein bleeding model. (A) Experimental scheme showing two temporal windows in which bleeding and clotting were monitored: 1.) with aptamer/PBS and no antidote (first 15-min time slot) followed by 2.) aptamer/PBS+/−antidote (second-15 min time slot) during which the number of times clots were formed (bleeding stopped) in transected murine vessels. (B) In vivo anticoagulant reversibility of POEGMA-RB005 with antidote oligonucleotide RB007 given at 10:1 molar excess. All animals were initially treated with either PBS vehicle or the POEGMA-RB005 (dose 2.0 mg kg⁻¹) in a murine saphenous vein-bleeding model. The vehicle group stopped bleeding 18±2 times over the initial 15 min (n=5), and then 22±1 times in the subsequent 15-min observation window. In contrast, animals receiving POEGMA-RB005 stopped bleeding 3±1 times initially demonstrating potent anticoagulation. Adding the specific antidote (RB007) 15 min after POEGMA-RB005 administration and measuring clot formation in a subsequent 15-min time window reveals a reversal of bleeding, with 20±1 clots formed, which was similar to animals that never received the aptamer (n=5). The POEGMA-RB005 group given an unrelated but equivalent size control antidote continued to bleed and demonstrated little to no clot development to limit hemorrhage (n=5). Error bars represent mean±SEM. Kolmogorov-Smirnov test (KS test) was used for each comparison: column A versus B, p=not significant (ns); column C versus D, p<0.01; column E versus F, p=not significant (ns); column A versus C, p<0.01; column D versus F, p<0.01. (Statistical significance: *p<0.05, **p<0.01).

FIG. 14 shows POEGMA-RB005 does not bind to patient-derived PEG antibodies. Competitive ELISA was performed using a patient-derived PEG antibody positive plasma sample. A dilution series of RB006, and RB005-POEGMA were mixed with a constant concentration of a human plasma sample that contains PEG antibodies and competed with a uricase-PEG conjugate adsorbed on the well-plate surface for binding to the PEG antibodies. Adenosine deaminase (ADA), exendin, and RB005 were used as PEG-minus negative controls. ADAgen was used as a PEGylated positive control. The data were analyzed using multiple unpaired t-tests between the PEGylated drug and its POEGMA counterpart. Data showed the mean±standard deviation of the mean. A statistical test was considered significant when the p<0.05 (*p<0.05; **p<0.01; ***p<0.001; **** p<0.0001). Not significant (ns). All statistical analyses were performed using GraphPad Prism 9.0. RB005, RB006, ADA, and ADAgen.

FIG. 15 shows analysis of PEG-specific ADA response over time. OVA-PEG- and OVA-POEGMA-coupled beads were used to determine ADAs induced towards PEG or POEGMA, respectively. The OVA-coupled bead was used as a control for cross-reactivity. ADA response (n=6) was measured for each mouse. Data represent the mean ADA response in a treatment group (n=10) and standard error of the mean (SEM). Data were analyzed by two-way repeated-measures ANOVA followed by post-hoc Tukey's multiple comparison test. Data were considered statistically significant when p<0.05. Not significant (ns). ****p<0.0001.

FIG. 16 shows RB005 variants do not induce an anti-PEG or anti-POEGMA IgG response. IgG response on (A) Day −7, (B) Day 14, (C) Day 35, and (D) Day 56. The sample size is n=10 mice per group. OVA-PEG- and OVA-POEGMA-coupled beads were used to determine ADAs induced towards PEG or POEGMA, respectively. ADA response (n=6) was measured for each mouse. Data represented the mean ADA response in a treatment group and standard error of the mean (SEM). Data were analyzed by two-way repeated-measures ANOVA followed by post-hoc Tukey's multiple comparison test. Data were considered statistically significant when p<0.05. Not significant (ns). ****p<0.0001.

### DETAILED DESCRIPTION

RNA aptamers are of significant therapeutic and clinical interest because their activity can be easily reversed in vivo—a useful feature that is difficult to achieve using other therapeutic modalities, such as antibodies. Despite their therapeutic promise, RNA aptamers are limited by their poor blood circulation. The attachment of polyethylene glycol (PEG) to RNA aptamers can address this limitation. However, an RNA aptamer-PEG conjugate that is a reversible anticoagulant failed in a clinical trial due to the reactivity of the conjugate with pre-existing PEG antibodies, leading to anaphylactoid responses.

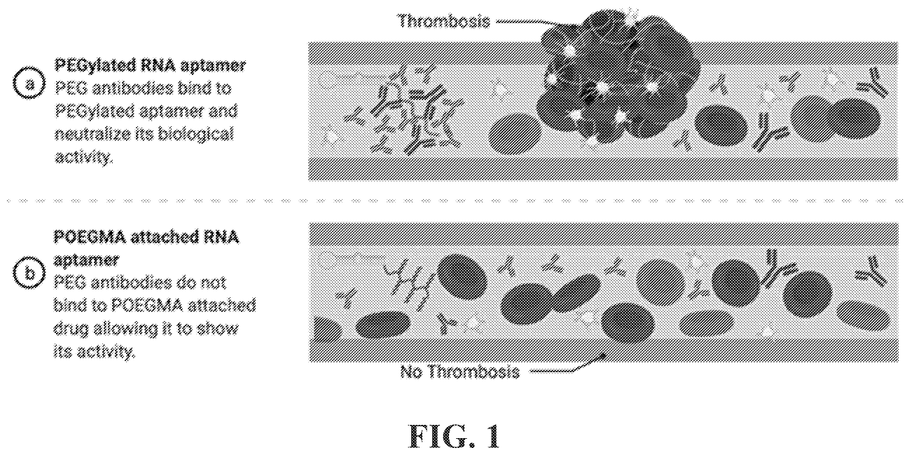

Here, PEG antibody-reactivity is eliminated for an RNA aptamer by conjugating it to a PEG-like brush polymer-poly[(oligoethylene glycol) methyl ether methacrylate)] (POEGMA) (FIG. 1). Significantly, the conjugate retained the aptamer's therapeutic action and the ability to be easily reversed. In addition, the conjugate does not bind pre-existing PEG antibodies that are prevalent in humans as measured in vitro and does not induce a humoral immune response against the polymer itself in mice. These findings suggest a path to rescuing the PEGylation of RNA therapeutics and vaccines from the deleterious side-effects of PEG.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the disclosed invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

The term “aptamer” refers to short, single-stranded oligonucleotides that can form a three-dimensional structure and bind to a target molecule with high affinity and specificity. Target molecules can include, but are not limited to, proteins, peptides, carbohydrates, small molecules, toxins, and cells.

The specificity of the aptamer binding is defined in terms of the comparative dissociation constants (K_{d}) of the aptamer for a target as compared to the dissociation constant with respect to the aptamer and other materials in the environment or unrelated molecules in general. Typically, the K_{d} for the aptamer with respect to the target will be 10-fold, 50-fold, 100-fold, or 200-fold less than the K_{d} with respect to the unrelated material or accompanying material in the environment.

An aptamer includes a 5′ end and a 3′ end. Aptamers are said to have “5′ ends” and “3′ ends” because nucleotides are reacted to make aptamers in a manner such that the 5′ phosphate of one nucleotide pentose ring is attached to the 3′ oxygen of its neighbor in one direction via a phosphodiester linkage. Thus, an end of an aptamer can be referred to as the “5′ end” if its 5′ phosphate is not linked to the 3′ oxygen of a nucleotide pentose ring and as the “3′ end” if its 3′ oxygen is not linked to a 5′ phosphate of a subsequent nucleotide pentose ring.

The term “antidote” refers to any pharmaceutically acceptable agent that can bind an aptamer and modify the interaction between that aptamer and its target molecule (e.g., by modifying the structure of the aptamer). An example antidote is an oligonucleotide that can bind to an aptamer and can change the three-dimensional configuration of the aptamer so that the aptamer can no longer interact with its target. The antidote oligonucleotide can be complimentary to a portion of the aptamer. The antidote oligonucleotide can change the conformation of the aptamer to reduce the target binding capacity of the aptamer by 10% to 100%, 20% to 100%, 25%, 40%, 50%, 60%, 70%, 80%, 90% or 100%, or any percentage in the range between 10% and 100% under physiological conditions. The antidote oligonucleotide can also form a three-dimensional structure with binding activity to a target molecule. This target can be the same or different from the target of the aptamer.

The term “antigen” refers to a molecule capable of being bound by an antibody or a T cell receptor. The term “antigen” also encompasses T-cell epitopes. An antigen is additionally capable of being recognized by the immune system and/or being capable of inducing a humoral immune response and/or cellular immune response leading to the activation of B-lymphocytes and/or T-lymphocytes. In some embodiments, the antigen contains or is linked to a Th cell epitope. An antigen can have one or more epitopes (B-epitopes and T-epitopes). Antigens may include polypeptides, polynucleotides, carbohydrates, lipids, small molecules, polymers, polymer conjugates, and combinations thereof. Antigens may also be mixtures of several individual antigens.

The term “antigenicity” refers to the ability of an antigen to specifically bind to a T cell receptor or antibody and includes the reactivity of an antigen toward pre-existing antibodies in a subject.

The terms “effective amount” or “therapeutically effective amount” refer to the amount of a conjugate or composition thereof that, when administered to a subject for preventing or treating thrombosis is sufficient to affect a treatment. An appropriate “effective” amount in any individual case may be determined using techniques, such as a dose escalation study. The dose could be administered in one or more administrations. However, the precise determination of what would be considered an effective dose may be based on factors individual to each patient, including, but not limited to, the patient's age, size, type or extent of disease, stage of the disease, route of administration, the type or extent of supplemental therapy used, ongoing disease process and type of treatment desired (e.g., aggressive vs. conventional treatment).

The term “immunogenicity” refers to the ability of any antigen to induce an immune response and includes the intrinsic ability of an antigen to generate antibodies in a subject. As used herein, the terms “antigenicity” and “immunogenicity” refer to different aspects of the immune system and are not interchangeable.

The term “subject” includes humans and mammals (e.g., mice, rats, pigs, cats, dogs, and horses). Typical subjects of the present disclosure may include mammals, particularly primates, and especially humans. For veterinary applications, suitable subjects may include, for example, livestock such as cattle, sheep, goats, cows, swine, and the like; poultry such as chickens, ducks, geese, turkeys, and the like, as well as domesticated animals particularly pets such as dogs and cats. For research applications, suitable subjects may include mammals, such as rodents (e.g., mice, rats, hamsters), rabbits, primates, and swine such as inbred pigs and the like.

The terms “treatment” or “treating,” as used herein, refer to protection of a subject from thrombosis related to a disease or disorder (which includes potential thrombosis related to surgical intervention of a disease or disorder), and means preventing, suppressing, repressing, ameliorating, or eliminating the thrombosis related to the disease or disorder. For example, preventing the thrombosis related to the disease or disorder involves administering a conjugate of the present disclosure, which can act as an anticoagulant, to a subject prior to onset of the disease.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 2. CONJUGATES

Disclosed herein are conjugates that include an aptamer and POEGMA conjugated to the aptamer. It has been found that replacing the PEG of a PEG-aptamer conjugate with POEGMA can reduce or eliminate the immune response directed to the POEGMA-aptamer conjugate compared to the PEG-aptamer conjugate. The reduced or eliminated immune response can include both a reduced or eliminated antigenicity and a reduced or eliminated immunogenicity of the POEGMA-aptamer conjugate. Accordingly, the disclosed conjugate can have beneficial interactions with a subject's immune system.

With respect to the PEG-aptamer conjugate, this molecule can be considered a control as to what the disclosed conjugate is compared to when assessing reducing or eliminating antigenicity, immunogenicity, or both. The control can be of similar molecular weight. The control can also be branched or linear, as long as it has more than the disclosed number of consecutive ethylene glycol monomers in tandem. For example, a suitable control PEG can include linear or branched PEG having more than 6 consecutive ethylene glycol monomers in tandem.

The beneficial immune interactions of the conjugate can also be seen in that the conjugate may not induce an anti-POEGMA antibody response, an anti-conjugate antibody response, an anti-aptamer antibody response, or a combination thereof. In some embodiments, the conjugate does not induce an anti-POEGMA antibody response. In addition, in some embodiments, the conjugate is not reactive with pre-existing anti-PEG antibodies in a subject.

The conjugate may have a varying hydrodynamic size due, in part, to the aptamer and POEGMA. For example, the conjugate can have a hydrodynamic size of about 4 nm to about 10 nm, such as about 4 nm to about 8 nm or about 4 nm to about 7 nm. In some embodiments, the conjugate has a hydrodynamic size of greater than about 3.6 nm, which can be useful to avoid renal excretion. Hydrodynamic size can be measured by techniques used within the art, such as dynamic light scattering.

A. Aptamers

The aptamer is not generally limited and can be any aptamer that can be conjugated to the POEGMA. The aptamer can localize to a specific target molecule, protein, cell, tissue, or the like. Accordingly, description regarding the binding of a target molecule by the aptamer can also be applied to the conjugate thereof. In some embodiments, the aptamer portion is capable of binding to a specific target protein. The target protein can be a cell surface protein, a protein present intracellularly, or a protein present extracellularly. In some embodiments, the target protein is a blood protein. A “blood protein,” also referred to as a “plasma protein,” is a protein found in blood plasma. In some embodiments, the target protein is a protein involved with the blood coagulation cascade.

In some embodiments, the target protein includes Factor IX (FIX) or the cleavage product Factor IXa (FIXa). In some embodiments, the aptamer binds to the complex formed by FIXa with Factor VIIIa (FVIIIa), also known as the “intrinsic tenase complex.” In some embodiments, the aptamer inhibits the complex formation between FIXa and FVIIIa. In some embodiments, the aptamer binds to the complex of FIX and FVIIIa and inhibits activation of Factor X (FX). The aptamer can interact with FIX, FIXa or a complex formed with FVIIIa in the presence or absence of additional calcium. The aptamer can also interact with the factors of the complex at a cell membrane. In some embodiments, the aptamer binds to the intrinsic tenase complex at the membrane surface.

The aptamer may include DNA, RNA, or both. The aptamer may include modified, e.g., chemically modified, nucleotides that can be useful for nuclease resistance, plasma stability, or a combination thereof. Examples of modified nucleotides includes, but are not limited to, nucleotides that have a sugar modified with 2′fluoro or 2′-O-methyl groups, are inverted, have a bioorthogonal functional group, and combinations thereof.

In some embodiments, the aptamer includes greater than 10% modified nucleotides, greater than 20% modified nucleotides, greater than 30% modified nucleotides, greater than 40% modified nucleotides, greater than 50% modified nucleotides, greater than 60% modified nucleotides, greater than 70% modified nucleotides, greater than 80% modified nucleotides, greater than 90% modified nucleotides, or greater than 95% modified nucleotides. In some embodiments, the aptamer includes less than 99% modified nucleotides, less than 95% modified nucleotides, less than 90% modified nucleotides, less than 80% modified nucleotides, less than 70% modified nucleotides, less than 60% modified nucleotides, less than 50% modified nucleotides, less than 40% modified nucleotides, less than 30% modified nucleotides, or less than 20% modified nucleotides. Percentages are based on total number of nucleotides.

The aptamer may have about 15 nucleotides to about 100 nucleotides, such as about 20 nucleotides to about 90 nucleotides, about 25 nucleotides to about 75 nucleotides, about 25 nucleotides to about 60 nucleotides, about 20 nucleotides to about 60 nucleotides, about 25 nucleotides to about 50 nucleotides, or about 25 nucleotides to about 40 nucleotides.

The aptamer may have varying structure depending on, e.g., its binding target. For example, the aptamer may include at least one region that binds to another region in the target molecule via Watson-Crick base pairing (stem) and at least one region that does not bind to any other regions of the target, e.g., under physiological conditions (loop). In some embodiments, the aptamer includes at least one stem and at least one loop. In some embodiments, the aptamer includes two stems (stem 1 and stem 2) and two loops (loop 1 and loop 2). In some embodiments, stem 1 is 1 to 20 nucleotides long. In some embodiments, stem 1 is 1 to 10 nucleotides long. In some embodiments, stem 1 is 7, 6, 5, 4, 3 or 2 nucleotides long. In some embodiments, stem 2 is 1 to 20 nucleotides long. In some embodiments, stem 2 is 1 to 10 nucleotides long. In some embodiments, stem 2 is 7, 6, 5, 4, 3, or 2 nucleotides long.

In some embodiments, the aptamer comprises SEQ ID NO: 1. In some embodiments, the aptamer consists of SEQ ID NO: 1.

i. Synthesis of Aptamers

The aptamers disclosed herein may be synthesized using methods well-known in the art. For example, the disclosed aptamers may be synthesized using standard phosphoramidite oligonucleotide synthesis technology employed by various commercial vendors including Integrated DNA Technologies, Inc, (IDT), Sigma-Aldrich, Life Technologies, or Bio-Synthesis, Inc. In addition, U.S. patents have issued describing methods of large-scale manufacturing that can be used to manufacture aptamers. Caruthers et al., for example, describe in U.S. Pat. Nos. 4,973,679; 4,668,777; and 4,415,732 a class of phosphoramidite compounds that are useful in the manufacture of oligonucleotides. In another series of patents, Caruthers et al. disclose a method of synthesizing oligonucleotides using an inorganic polymer support. See, e.g., U.S. Pat. Nos. 4,500,707, 4,458,066 and 5,153,319. In another series of patents, Caruthers et al. discloses a class of nucleoside phosphorodithioates that can be used to manufacture oligonucleotides. See, e.g., U.S. Pat. Nos. 5,278,302, 5,453,496 and 5,602,244. All of these patents are incorporated herein by reference in their entirety.

The manufacture of the disclosed aptamers can be a multi-step process involving solid phase chemical synthesis of the oligonucleotide strand; cleavage and deprotection of the crude oligonucleotide; and purification by preparative anion exchange chromatography, Reverse Phase HPLC or UPLC.

By employing the combinatorial in vitro selection method termed SELEX (Systematic Evolution of Ligands by Exponential Enrichment) (Tuerk and Gold, 1990, Ellington and Szostak, 1990, which are both incorporated by reference herein in their entirety) aptamers can be generated to a broad range of targets including proteins and small molecules. By utilizing a nucleic acid library of approximately 10¹⁴ different sequences containing a randomized stretch of nucleotides, the oligonucleotide pool can form a vast array of distinct secondary and tertiary structures. By screening this diverse RNA library through iterative rounds of binding, partitioning, and amplification, aptamers can be generated to targets of interest with high affinity and specificity.

Further discussion on aptamers, their synthesis, and their application can be found in U.S. Pat. Nos. 7,300,922 and 7,531,524, which are incorporated herein by reference in their entirety.

B. POEGMA

The POEGMA can instill the conjugate with advantageous stealth and immune system properties. In addition, the POEGMA can be conjugated to the aptamer while still retaining the aptamer's ability to reversibly bind to a target molecule. The POEGMA has a poly(methyl methacrylate) backbone and a plurality of side chains covalently attached to the backbone. The side chains are oligomers of ethylene glycol (EG). For example, each side chain can include about 1 to about 6 monomers of EG repeated in tandem, such as about 1 to about 5 monomers of EG repeated in tandem, about 2 to about 4 monomers of EG repeated in tandem, or about 1 to about 3 monomers of EG repeated in tandem. In some embodiments, each side chain includes about 3 monomers of EG repeated in tandem.

Adjacent side chains may be the same within the same POEGMA molecule or they may be different. For example, one side chain may have 2 monomers of EG repeated in tandem, while another side chain (in the same POEGMA molecule) may have 3 monomers of EG repeated in tandem. Each side chain has a first terminal end and a second terminal end. The first terminal end is covalently attached to the backbone. The second terminal end can be free. The second terminal end may be modified. In some embodiments, the second terminal end independently comprises an alkyl, ester, amine, amide, or carboxyl group. In some embodiments, the second terminal end of each side chain does not include a hydroxyl group. The second terminal end of each side chain may be the same or different from the second terminal end of an adjacent side chain in the same POEGMA molecule. In some embodiments, the second terminal end of each side chain is the same throughout the POEGMA. In some embodiments, the second terminal end of at least one side chain is different from the second terminal end of at least one adjacent side chain.

The POEGMA can have a varying molecular weight. For example, the POEGMA can have a number average molecular weight of about 5 kDa to about 50 kDa, such as about 10 kDa to about 40 kDa, about 5 kDa to about 40 kDa, or about 25 kDa to about 45 kDa. Molecular weight of the POEGMA can be measured by techniques used within the art, such as size-exclusion chromatography (SEC), SEC combined with multi-angle light scattering, gel permeation chromatography, and the like.

Further discussion on POEGMA, its synthesis, and it application can be found in U.S. Pat. Nos. 8,497,356 and 10,364,451, both of which are incorporated herein by reference in their entirety.

C. Methods of Making Conjugates

Also disclosed are methods of making the conjugates. The method can include conjugating the POEGMA having a poly(methyl methacrylate) backbone and a plurality of side chains covalently attached to the backbone, each side chain comprising 1 to 6 monomers of ethylene glycol (EG) repeated in tandem to the aptamer to provide the conjugate. The POEGMA can be conjugated to the 5′ end or the 3′ end of the aptamer. In some embodiments, the POEGMA is conjugated to the 5′ end aptamer.

The POEGMA can be conjugated to the aptamer by conjugation strategies known within the art. For example, the aptamer and the POEGMA may each individually have functional groups that are complimentary to each other in that they can form a covalent bond between the functional groups under appropriate conditions. Representative complimentary functional groups that can form a covalent bond include, but are not limited to, an amine and an activated ester, an amine and an isocyanate, an amine and an isothiocyanate, thiols for formation of disulfides, an aldehyde and amine for enamine formation, an azide for formation of an amide via a Staudinger ligation. Functional groups suitable for conjugation also include bioorthogonal functional groups. Bioorthogonal functional groups can selectively react with a complementary bioorthogonal functional group. Bioorthogonal functional groups include, but are not limited to, an azide and alkyne for formation of a triazole via Click-chemistry reactions, trans-cyclooctene (TCO) and tetrazine (Tz) (e.g., 1,2,4,5-tetrazine), and others. In some embodiments, the aptamer and the POEGMA each individually include bioorthogonal functional groups. In some embodiments, the aptamer is functionalized with dibenzocyclooctyne, the POEGMA is functionalized with an azide, or both.

The aptamer can be conjugated to the POEGMA while maintaining its ability to bind to specific targets. In addition, the aptamer can be conjugated to the POEGMA while maintaining its ability to be neutralized by an antidote oligonucleotide. If a potential conjugation technique results in a loss of efficacy of the aptamer binding to its target, numerous modifications within the art can be tried including, but not limited to, conjugating POEGMA to the 3′ end as opposed to the 5′ end, introducing a spacer (such as a C6, C12, Cis, etc. linker or other suitable chemical spacers) that can increase the distance between the aptamer and the POEGMA and still maintain the beneficial properties of the aptamer. Because aptamers are chemically synthesized, the addition of chemical moieties facilitating derivatization with POEGMA at numerous specific sites in the aptamer sequence is possible.

The description of the conjugates, aptamers, and POEGMA can also be applied to the methods of making disclosed herein.

### 3. USES OF THE CONJUGATES

The present disclosure also provides methods of controlling coagulation in a subject. Thrombosis is the primary cause of death worldwide. Despite the advances in developing safer antithrombotic agents, they are limited with the significant risk of bleeding. The bleeding risk is even higher for patients undergoing highly prothrombotic clinical procedures, including percutaneous coronary intervention, coronary artery bypass graft (CABG) surgery, and dialysis. The disclosed conjugates can be used to inhibit thrombosis in a subject. Together with an antidote capable of rapidly reversing the anticoagulant activity of the conjugate, the pair can precisely control coagulation in a subject.

The method can include administering to a subject in need thereof a therapeutically effective amount of the disclosed conjugate, wherein administering the conjugate prevents or reduces blood clot formation in the subject. “Preventing blood clot formation” may include reducing the likelihood of blood clots, reducing the size of blood clots or slowing further progression of blood clotting, where reducing is compared to the conjugate not being administered.

The disclosed conjugates can be administered to patients suffering from or at risk of suffering from a cardiovascular disease or intervention, including surgical intervention, that causes or results in a coagulation-inducing event. Examples include acute myocardial infarction (heart attack), cerebrovascular accidents (stroke), ischemia, angioplasty, CABG, cardiopulmonary bypass, thrombosis in the circuit of cardiac bypass apparatus and in patients undergoing renal dialysis, unstable angina, pulmonary embolism, deep vein thrombosis, arterial thrombosis, and disseminated intravascular coagulation. Other related examples include subjects suffering from FV Leiden or atrial fibrillation. The subject that can benefit from the disclosed methods can be any type of mammal. In some embodiments, the subject is human. In some embodiments, the subject is a patient preparing for, in, or coming out of surgery, e.g., a surgery patient.

It is also important to be able to release blood coagulation factors from inhibition. Life threatening diseases can result from over-inhibition of the blood coagulation factors such as Factor IX. Accordingly, disclosed herein is an antidote paired with the aptamer. Antidotes can include any pharmaceutically acceptable agent that can bind an aptamer and modify the interaction between that aptamer and its target molecule (e.g., by modifying the structure of the aptamer). Examples of such antidotes include (A) oligonucleotides complementary to at least a portion of the aptamer sequence (including ribozymes or DNAzymes or peptide nucleic acids (PNAs)), (B) nucleic acid binding peptides, polypeptides or proteins (including nucleic acid binding tripeptides (see, generally, Hwang et al. (1999) Proc. Natl. Acad. Sci. USA 96:12997), and (C) oligosaccharides (e.g. aminoglycosides (see, generally, Davis et al. (1993) Chapter 8, p. 185, RNA World, Cold Spring Harbor Laboratory Press, eds. Gestlaad and Atkins; Werstuck et al. (1998) Science 282:296; U.S. Pat. Nos. 5,935,776 and 5,534,408). (See also the following which disclose types of antidotes that can be used herein: Chase et al. (1986) Ann. Rev. Biochem. 56:103, Eichorn et al. (1968) J. Am. Chem. Soc. 90:7323, Dale et al. (1975) Biochemistry 14:2447 and Lippard et al. (1978) Acc. Chem. Res. 11:211). In some embodiments, the antidote is an antidote oligonucleotide.

The antidote oligonucleotide can neutralize the conjugate when administered at a therapeutically effective amount. Generally, a therapeutically effective amount, as it relates to the antidote oligonucleotide, is an amount of the antidote oligonucleotide sufficient to produce a measurable modulation of the effects of the aptamer or conjugate thereof, including but not limited to a coagulation-modulating amount. In some embodiments, the antidote oligonucleotide reverses or neutralizes at least 25%, 50%, 75%, 80% or 90% of the anticoagulant activity of the aptamer and conjugate thereof. The antidote generally has the ability to substantially bind to an aptamer in solution at antidote concentrations of less than 1 μM, less than 0.1 μM or less than 0.01 M. In some embodiments, the antidote oligonucleotide reduces the biological activity of the aptamer and conjugate thereof by about 50%. The antidote oligonucleotide can also be used to modulate the function of the aptamer if only partial aptamer activity is desired. For example, the aptamer's activity can be “fine-tuned” as needed for different applications and/or diseases. Or, in some embodiments, complete reversal of aptamer activity can be achieved.

In some embodiments, the antidote oligonucleotide includes SEQ ID NO: 2. In some embodiments, the antidote oligonucleotide consists of SEQ ID NO: 2.

The description of the conjugates, aptamers, and POEGMA can also be applied to the uses disclosed herein.

### 4. ADMINISTRATION

The disclosed conjugates may be incorporated into pharmaceutical compositions suitable for administration to a subject (such as a patient, which may be a human or non-human) well known to those skilled in the pharmaceutical art. The pharmaceutical composition may be prepared for administration to a subject. Such pharmaceutical compositions can be administered in dosages and by techniques well known to those skilled in the medical arts taking into consideration such factors as the age, sex, weight, and condition of the particular subject, and the route of administration.

The pharmaceutical compositions may include pharmaceutically acceptable carriers. The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols, such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline: Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator. The route by which the composition is administered and the form of the composition will dictate the type of carrier to be used.

The composition can be administered prophylactically or therapeutically. The compositions can be administered by methods well known in the art as described in Donnelly et al. (Ann. Rev. Immunol. 1997, 15, 617-648); Felgner et al. (U.S. Pat. No. 5,580,859, issued Dec. 3, 1996); Felgner (U.S. Pat. No. 5,703,055, issued Dec. 30, 1997); and Carson et al. (U.S. Pat. No. 5,679,647, issued Oct. 21, 1997), which are all incorporated by reference herein in their entirety. One skilled in the art would know that the choice of a pharmaceutically acceptable carrier, including a physiologically acceptable compound, depends, for example, on the route of administration. The compositions can be delivered via a variety of routes. Typical delivery routes include parenteral administration, e.g., intradermal, intramuscular or subcutaneous delivery. Other routes include oral administration, intranasal, intravaginal, transdermal, intravenous, intraarterial, intratumoral, intraperitoneal, and epidermal routes. In some embodiments, the composition is administered intravenously to the subject.

The disclosed invention has multiple aspects, illustrated by the following non-limiting examples.

### 5. EXAMPLES

### Example 1

### POEGMA Conjugated Aptamer

### Materials & Methods

POEGMA synthesis and purification: Triethylene glycol methyl ether methacrylate (EG3; Millipore Sigma) monomer was purified from polymerization inhibitors by passing it through a basic alumina column. 2-Azidoethyl 2-bromoisobutyrate (Millipore Sigma) was used as the polymerization initiator. Tris(2-pyridylmethyl) amine (TPMA; Millipore Sigma) was mixed with copper (II) bromide (CuBr₂; Alfa Aeasar) in water at a 1:8 molar ratio (0.1M:0.8M), yielding the catalytic complex. Inhibitor-free EG3 (10 mmol; 232.3 mg; 2.262 ml) was mixed with the polymerization initiator (0.05 mmol; 11.8 mg) and the catalytic complex (125 μl; 0.1 mmol TPMA; 0.013 mmol CuBr₂) in a 30% (vol) methanol (6 ml) mixture of 100 mM sodium chloride (NaCl; 11.613 ml) solution in the polymerization flask. The resulting mixture (20 ml) was sealed and purged with argon for 1 h. In a separate flask, ascorbic acid (Millipore Sigma) was dissolved in 100 mM NaCl at a final concentration of 64 mM and was then purged with argon for 1 h. The polymerization was carried out by continuously injecting ascorbic acid into the polymerization flask at a rate of 1 μl min⁻¹ for 2 h under an inert argon atmosphere. The polymerization was stopped by exposing the solution to air. The polymerization mixture was dialyzed into water for three days using a dialysis membrane with a molecular-weight cut-off (MWCO) of 3000 Da, followed by lyophilization.

DBCO-RB005 synthesis and purification: RB005 was reacted with N-hydroxy succinimide (NHS) ester functionalized DBCO (Click Chemistry Tools), yielding DBCO-RB005. Briefly, 1.6 μmol RB005 (16.6 mg) was dissolved in dimethylsulfoxide (DMSO; 200 μl) and 0.1 M borate buffer (480 μl), followed by the addition of DBCO-NHS ester (160 μmol; 85.9 mg) and allowing the mixture to react for 1 h at 30° C. The reaction mixture was desalted using a Zeba desalting column (Pierce) with a 7 kDa MWCO to remove unreacted DBCO-NHS ester, and the product was lyophilized.

Conjugate synthesis and purification: POEGMA-RB005 was synthesized by a strain promoted alkyne-azide click reaction using DBCO-RB005 and azide functional POEGMA as the reactants. Briefly, DBCO-RB005 (1.6 μmol; 17.3 mg; 2.54 mM in 630 μl water) was reacted with azide functional POEGMA (2 μmol; 78.6 mg; 3.18 mM in 70 μl acetonitrile) for 24 h at room temperature. For the synthesis of RB006, RB005 (1.6 μmol; 16.6 mg) was dissolved in DMSO (200 μl) and 0.1 M borate buffer at pH 8 (280 μl) and reacted with an NHS ester functionalized 2-arm branched PEG (Sunbright; NOF) (2.4 μmol; 102 mg; 326.4 μl in 0.1 M borate buffer at pH 8) for 2 h at room temperature. The resulting conjugates were purified using anion exchange chromatography using an AKTA Purifier (GE Healthcare) equipped with a photodiode array operating at 220 and 280 nm, a HiPrep Q HP (GE Healthcare) column, and 20 mM ammonium carbonate pH 8.2 as binding buffer. Unreacted POEGMA and PEG were collected in the flow through, while the conjugates were collected using gradient elution with 0.5 M NaCl in the binding buffer. The conjugates were desalted using a Zeba desalting column (Pierce) with a 7 kDa MWCO, followed by lyophilization.

Physical characterization: TBE-Urea gel electrophoresis was performed using pre-cast Mini-Protean TBE-urea gels (Biorad) according to manufacturers' protocols. Briefly, the aptamer samples were denatured at 95° C. for 5 minutes, followed by loading to the gel and running it until the dye front reaches the reference line. RiboReady™ Color Micro RNA Ladder was used for size comparison.

The Mₙ, M_{w}, and Ð of the conjugates and polymers were measured by SEC-MALS. The compounds were solubilized in the mobile phase (20 mM phosphate buffer (pH 7.2) supplemented with NaCl (50 mM) and MgCl₂ (3 mM)) and filtered through a 100 nm syringe filter (Whatman Anotop). The compounds were separated on a Shodex KW-803 column (8 mm×300 mm; 5 μm) using an Agilent 1260 analytical HPLC equipped with UV (Agilent), a DAWN HELEOS II MALS (Wyatt Technology), and Optilab T-rEX refractive index detectors (Wyatt Technology). Toluene was used for annual calibration of MALS, and bovine serum albumin was used for detector normalization prior to each SEC-MALS run. The flow rate was 0.5 ml min⁻¹. ASTRA software (v. 8.0, Wyatt Technology) was used for data analysis.

Hydrodynamic size characterization: The Rₕ of conjugates, polymers, and intermediates were measured using DLS. The compounds were solubilized in PBS and filtered through a 100 nm syringe filter (Whatman Anotop). Data were acquired at 15° C. using DynaPro Plate Reader (Wyatt Technology) and analyzed for Raleigh spheres using Dynamics 6.12.0.3 software (Wyatt Technology).

Anticoagulant activity characterization: The anticoagulant activity of the conjugates was tested in an aPTT assay. The assay was performed using a Stago STart 4 mechanical coagulometer. Serial dilutions of RB006 and POEGMA-RB005 were made in PBS without divalent cations (Gibco Life Technologies). 50 μL of pooled normal human plasma (George King Bio-Medical), 5 μl of the diluted conjugate, and 50 μL of TriniCLOT aPTT S reagent (Tcoag) were incubated for 5 min at 37° C. Following the incubation, coagulation was initiated with 50 μL of TriniCLOT 0.02 M calcium chloride solution (Tcoag). Data were presented as the mean aPTT clotting time in seconds+SEM and plotted as a function of aptamer plasma concentration.

To ensure that POEGMA conjugation did not impact the ability of RB007 to reverse the function of the aptamer, a modified aPTT assay was performed. The aptamer reversal agent RB007 was synthesized utilizing standard solid phase phosphoramidite chemistry. The RB007 nucleotide sequence was mCmGmCmGmGmUmAmUmAmGmUmCmCmAmC, where mC, mG, m, and mA represent 2′O-methyl Cytosine, 2′O-methyl Guanosine, 2′O-methyl Uridine, and 2′O-methyl Adenosine, respectively. Fixed concentrations of both RB006 and POEGMA-RB005, and varying molar excesses of RB007 were diluted in PBS without divalent cations (Gibco Life Technologies). RB006 and POEGMA-RB005 concentrations were set at 392 nM final plasma concentration. 50 μL of pooled normal human plasma (George King Bio-Medical) and 5 μl of the diluted conjugate were incubated for 5 min at 37° C. After the incubation, 5 μL of reversal agent RB007 and 50 μL of TriniCLOT aPTT S reagent (Tcoag) were added, followed by a second 5-min incubation at 37° C. Coagulation was initiated by the addition of 50 μL of TriniCLOT 0.02 M calcium chloride solution (Tcoag).

Murine PEG antigenicity ELISA: The reactivity of POEGMA-RB006 and RB006 to murine PEG antibodies was tested in indirect and competitive ELISAs. The antigen solutions used for coating the wells yielded 4 μg PEG/POEGMA per well. The amount of protein/RNA aptamer per well varied depending on the overall conjugate M_{w} and stoichiometry, but kept constant among a conjugate group, such that the wells coated with RB005, RB006, and POEGMA-RB005 contained nearly constant amount of RB005.

In the indirect ELISA, the wells of 96-well microtiter plates (Corning) were coated with 100 μL of antigens —diluent, OVA-PEG, RB005, RB006, and POEGMA-RB005-overnight at 4° C. On the day of the assay, the antigens solutions were removed, and the wells were blocked with 1% iBlock (Thermo Scientific) in PBS for 1 h at room temperature. The wells were washed three times with PBS. An anti-PEG antibody-positive murine plasma sample was diluted in PBS (1:500 vol/vol), and 100 μL of the diluted plasma were transferred to the wells, followed by incubation for 1 h. The diluted plasma was removed, and the wells were washed with several rounds of PBS. 100 μl of a biotinylated anti-mouse IgM antibody (Jackson Immunoresearch; 115-065-075; 50 ng ml⁻¹) was transferred to the wells and incubated for 1 h. The wells were washed with PBS to remove the antibody, and 100 μl of streptavidin-poly HRP (Pierce; 0.1 μg ml⁻¹) was transferred to the wells, followed by incubation for 30 min. Excess HRP was removed with several rounds of PBS wash. 50 μl of TMB substrate solution (Pierce) were added to wells and incubated for 10 min. The TMB reaction was then stopped by adding 50 μl of 2 M sulfuric acid to each well, and the absorbance at 450 nm was measured on a Victor plate reader (Perkin Elmer).

In the competitive ELISA, the wells of a 96-well microtiter plate (Corning) were coated with 100 μL of an exendin-PEG conjugate (57.8 μg ml-1) overnight at 4° C. The exendin-PEG conjugate had an MW of 45.6 kDa and contained 40.4 g PEG per mole of the conjugate. An anti-PEG IgM-positive murine plasma sample, which was collected from C57BL/6J mice repeatedly treated with OVA-PEG in Freund's adjuvant, was available from a previous study. The plasma sample was diluted in PBS (1:250 vol/vol). 300 μL of the diluted plasma were mixed with 300 μL of the one of the following drugs—RB005, RB006, and POEGMA-RB005—at varied concentrations, followed by overnight incubation at 4° C. with end-to-end rotation. On the day of the assay, the solutions were removed, and the wells were blocked for 1 h, followed by two rounds of PBS wash. The drug: plasma mixtures were transferred to exendin-PEG coated wells and incubated for 1 h at room temperature on an orbital shaker. The drug: plasma mixtures were removed, and the wells were washed with several rounds of PBS. 100 μl of a biotinylated anti-mouse IgM antibody (Jackson Immunoresearch; 115-065-075; 50 ng ml-1) was transferred to the wells and incubated for 1 h. The wells were washed with PBS to remove the antibody, and 100 μl of streptavidin-poly HRP (Pierce; 0.1 μg ml-1) was transferred to the wells, followed by incubation for 30 min. Excess HRP was removed with several rounds of PBS wash. 50 μl of TMB substrate solution (Pierce) were added to wells and incubated for 10 min. The TMB reaction was then stopped by adding 50 μl of 2 M sulfuric acid to each well, and the absorbance at 450 nm was measured on a Victor plate reader (Perkin Elmer). The competitive ELISA with patient-derived PEG antibodies was performed as previously described in A. Moreno et al., Cell Chemical Biology 2019, 26, 634, which is incorporated by reference herein in its entirety.

Endotoxin purification and testing: The compounds were endotoxin tested using the Endosafe nexgen-PTS instrument and cartridges (Charles River) and sterilized using a sterile 0.22 sm syringe filter. The final endotoxin amount was kept below 0.2 EU per kg mouse body weight.

Aptamer refolding: Prior to animal studies, the conjugates were refolded in platelet-binding buffer (20 mM HEPES pH 7.4, 150 mM NaCl, 1 mM CaCl2, 1 mM MgCl2, and 5 mM KCl). Aptamer solutions were heated to 95° C. for 3 min, placed on ice for 3 min, and then allowed to come to room temperature over approximately 5-10 min.

Animal studies: The animal studies were carried out under the protocols approved by Duke University's Institutional Animal Care and Use Committee (IACUC). Investigators who performed surgery or analyzed carotid flow were blinded to the treatment groups. Eight-week-old female C57BL/6J mice were used in all experiments (Jackson Laboratories; #000664) after being acclimatized to the animal facilities for one week. The mice were group-housed in a light-controlled environment and had ad libitum water and food access.

Carotid Artery Injury: Mice were anesthetized with a combination of isoflurane and tribromoethanol (125 mg kg⁻¹; intraperitoneal). Mice were intubated and mechanically ventilated (Harvard Apparatus rodent ventilator, Holliston, MA), and then placed supine on a temperature-monitoring board. The right common carotid artery and left external jugular vein were isolated. The left jugular vein was then catheterized for all drug administration. Baseline carotid flow was obtained with a Doppler flow probe (Transonic Systems, Ithaca, NY) and LabChart computerized data acquisition program (ADInstruments, Colorado Springs, CO). Mice were treated with either negative control (platelet-binding buffer; 20 mM HEPES pH 7.4, 150 mM NaCl, 5 mM KCl, 1 mM MgCl₂, and 1 mM CaCl₂)) or the conjugates (1.0 to 2.0 mg kg⁻¹ in approximately 120 to 160 μL injection volume). The treatments and control were allowed to circulate for 5 min. Carotid artery injury was initiated by applying a 1.0×2.0 mm strip of Whatman No. 1 filter paper soaked in 10% FeCl₃ solution to the adventitial surface for 3 min. Blood flow was then measured for 60 min. The time to occlusion was recorded. Blood was collected for serum preparation, and the right carotid artery was harvested. Mice were then sacrificed.

Saphenous Vein Bleeding: Female 13—to 14-week-old C57BI/6J mice were obtained from Jackson Laboratory. Animals were anesthetized with a combination of isoflurane and tribromoethanol (125 mg kg⁻¹; intraperitoneal). The hair on the medial aspect of the right hind limb was removed. Mice were intubated and mechanically ventilated (Harvard Apparatus rodent ventilator, Holliston, MA), and placed supine on a temperature-monitoring board. The left external jugular vein was isolated and catheterized for all drug administration. The skin on the medial aspect of the right hind limb was incised, exposing a length of the saphenous neurovascular bundle; the bundle was covered with normal saline to prevent drying. Mice were treated with either negative control (PBS, phosphate buffered saline with calcium chloride and magnesium chloride) or aptamer conjugates (2.0 mg kg⁻¹). The treatments and control were allowed to circulate for 5 minutes. To assess hemostasis, the right saphenous vein was then transected by piercing it with a 23G needle, followed by a longitudinal incision made in the distal portion of the vessel. Blood was gently wicked away until hemostasis occurred. The clot was then removed to restart bleeding, and the blood was again wicked away until hemostasis occurred again. Clot disruption was repeated after every incidence of hemostasis for 15 minutes. Mice were then treated with either negative control (PBS), specific antidote oligonucleotide (RB007), or an unrelated antidote oligonucleotide control. The second treatments were allowed to circulate for 5 minutes, and hemostasis was then assessed again for 15 additional minutes, as described above. Mice were then sacrificed.

Immunogenicity: One week before initial dosing, a baseline blood sample was collected via submandibular bleeding with a 4 mm Goldenrod lancet (MEDIpoint, Mineola, NY). On days 0, 21, 42, animals were dosed by s.c. injection of either control (saline) or the conjugates (2.0 mg kg¹) in approximately 120 to 160 μL injection volume. On days 14, 35, and 56, a blood sample for serum preparation was collected via submandibular bleeding with a Goldenrod lancet. Blood sample was allowed to sit for 10-15 min to form a clot. The sample was then centrifuged at 4400 g for 10 min. The serum layer was isolated and stored at −80° C.

Luminex muliplexed immunoassays: ADAs were analyzed using a validated Luminex multiplexed assay as described in I. Ozer et al., Research Square, 2021, which is incorporated by reference herein in its entirety. The assay used fluorescently barcoded and drug-coupled magnetic microspheres to capture ADAs. Briefly, the serum samples were diluted 25-fold in PBS, and 50 μl of the diluted samples were transferred to a black 96-well-plate (n=6; Corning). The OVA-, OVA-PEG-, and OVA-POEGMA-coupled bead sets were mixed, such that the mixture contained 2500 beads per 50 μl per bead set. The resulting bead mixture was transferred into the wells and incubated for 1 h at room temperature on a plate shaker. Mouse IgG or IgM coupled beads were used as positive controls and incubated with the assay diluent (1% iBlock in PBS). The drug-coupled beads incubated in the assay diluent were used as negative controls. The plate was placed on a magnetic stand for 1 min to separate the magnetic microspheres. The beads were washed with several rounds of PBS to remove unbound proteins. Next, the wells containing the magnetic microspheres were incubated with 100 μl of biotin-conjugated goat anti-mouse IgM (Jackson Immunoresearch; #115-065-075; 5 μg ml⁻¹) or R-Phycoerythrin-conjugated goat anti-mouse IgG (Jackson Immunoresearch; #115-115-164; 5 μg ml⁻¹) incubation for 1 h at room temperature. The supernatant was discarded, and the wells treated with the anti-mouse IgM antibody were incubated with streptavidin-R-phycoerythrin (Invitrogen; 100 μl; 7.5 μg ml⁻¹) for 30 min. The wells were washed with PBS. The magnetic beads were solubilized in 100 μl of the assay diluent. The beads were analyzed using a MAGPIX (Luminex). The fluorescence intensity derived from each bead set in each mouse plasma sample was normalized to the signal measured with the positive control.

Statistical Analysis: Mice were randomly distributed into groups for all in vivo experiments using random.org. The group sizes were estimated using the open-source G-Power software based on literature studies and increased when necessary to ensure adequate power to detect differences. In our experience, even n=3 was often enough to see statistically significant differences in the PD, while n=5-6 was deemed sufficient for the immunogenicity experiments. The experiments were replicated at least twice with at least 3 repeats, unless otherwise stated. Data were presented as the mean±standard error of the mean, unless otherwise stated. The aPTT data were analyzed using an unpaired t-test. Other data were analyzed using two-way repeated measures ANOVA, followed post-hoc Dunnett's or Tukey's multiple comparison tests. A statistical test was considered significant for p<0.05 (*p<0.05; **p<⁰0.01; ***p<0.001; ****p<0.0001). Not significant (ns). All statistical analyses were performed using GraphPad Prism 9.0.

### Results

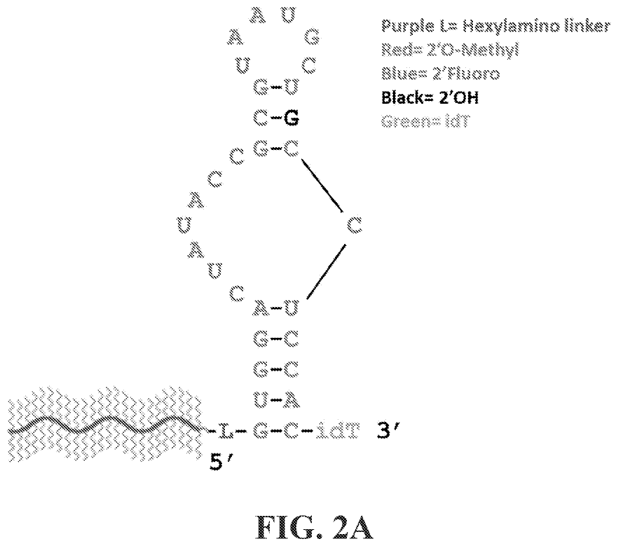

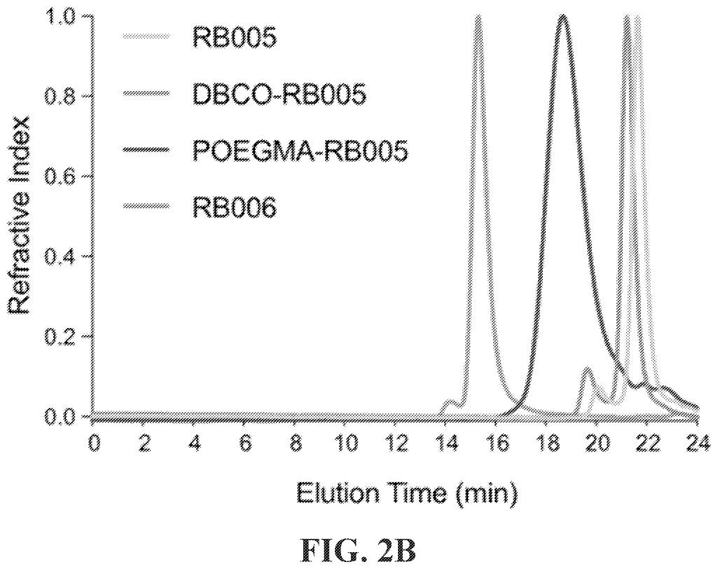

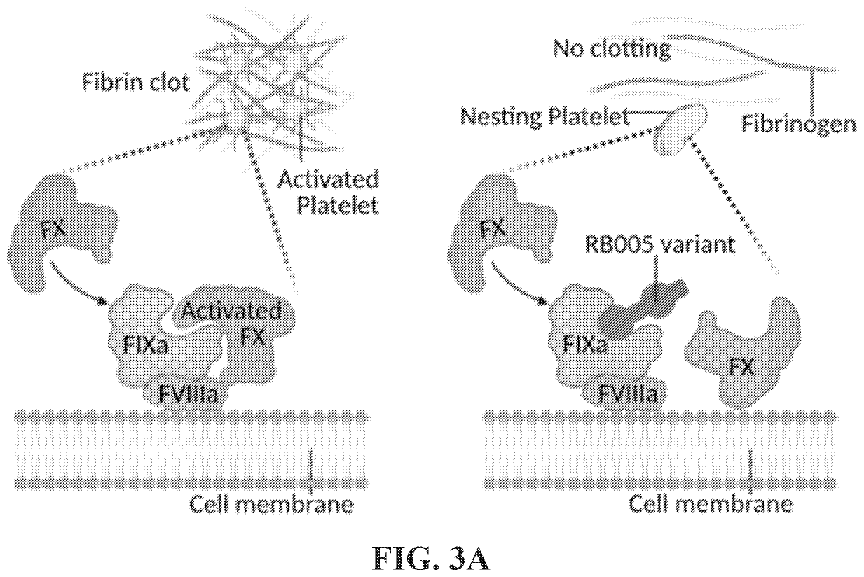

Conjugate Synthesis and Characterization: POEGMA can preserve PEG's pharmacokinetic (PK) benefits while simultaneously eliminating its reactivity towards PEG antibodies and being itself non-immunogenic. We exploited these favorable features of POEGMA to eliminate pegnivacogin's PEG antigenicity by formulating the parent FIXa-binding aptamer in pegnivacogin—termed RB005—as a POEGMA conjugate. RB005 is a 31-nucleotide RNA aptamer synthesized using chemically modified nucleotides to confer serum stability to the aptamer (FIG. 2A). The majority of ribose sugars are modified with 2′-fluoro or 2′-O-methyl groups to enhance nuclease resistance and eliminate the inherent immunogenicity of unmodified RNA. The 3′-end was modified with an inverted deoxythymidine residue to protect the aptamer against exonuclease attack. The 5′-end was modified with a hexylamino linker to provide a site for POEGMA or PEG conjugation. A note on nomenclature: the drug developed by Regado Biosciences that failed its Phase 3 clinical trial is termed pegnivacogin or RB006 and includes aptamer RB005 conjugated to a 2-arm branched PEG (NOF) with a M_{w} of 40 kDa, whereas the POEGMA conjugate is termed POEGMA-RB005. We synthesized a biosimilar —termed RB006—which includes RB005 conjugated to the same branched PEG used in the synthesis of pegnivacogin as a control.

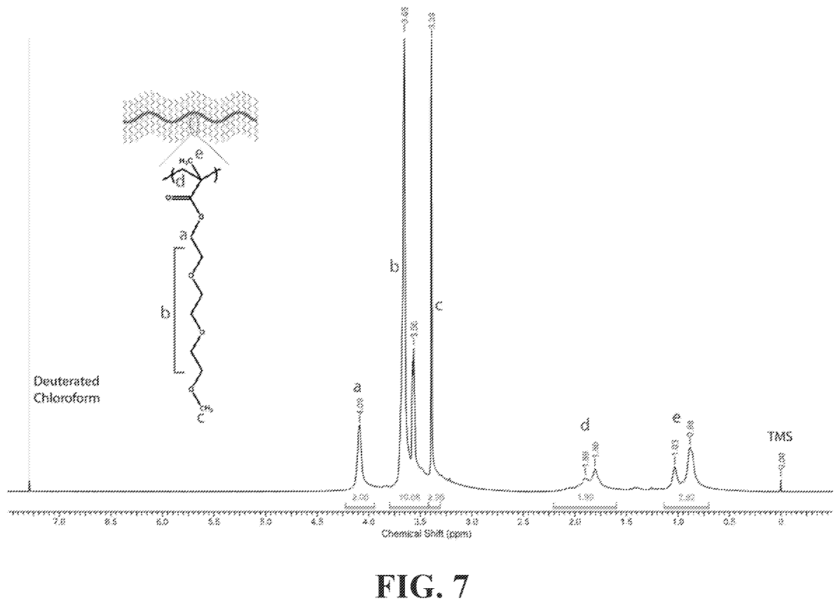

We synthesized an azide-functional POEGMA using activator-regenerated by electron transfer atom transfer radical polymerization (ARGET-ATRP). We chose a monomer with an oligo ethylene glycol (OEG) side-chain length of three, as confirmed by Nuclear Magnetic Resonance (NMR) spectroscopy (FIG. 7), because it lacks the antigenic epitope, while longer OEG chains still show some degree of reactivity to PEG antibodies. The synthesized POEGMA was well-defined, with a weight-average molecular weight (M_{w}) of ˜40 kDa, and had a low polydispersity (D) of <1.1 (Table 1), as measured by size exclusion chromatography-multi-angle light scattering (SEC-MALS). This M_{w} was chosen because a branched PEG with an M_{w} of 40 kDa was initially used for synthesis of pegnivacogin, and because POEGMA of this M_{w} is slowly cleared by renal excretion.

We next synthesized a POEGMA-reactive derivative of RB005. We converted the amine-functional 5′-end of RB005 to a dibenzocyclooctyne (DBCO) group, yielding DBCO-RB005. The bioorthogonal DBCO group was chosen because it should readily react with the terminal azide group in POEGMA via strain-promoted alkyne-azide click reaction without reacting with any other chemical groups on RB005, yielding a site-specific and stoichiometric (1:1) POEGMA-RB005 conjugate. We also synthesized a M_{w}-matched branched PEG conjugate of RB005—termed RB006—to use as a control

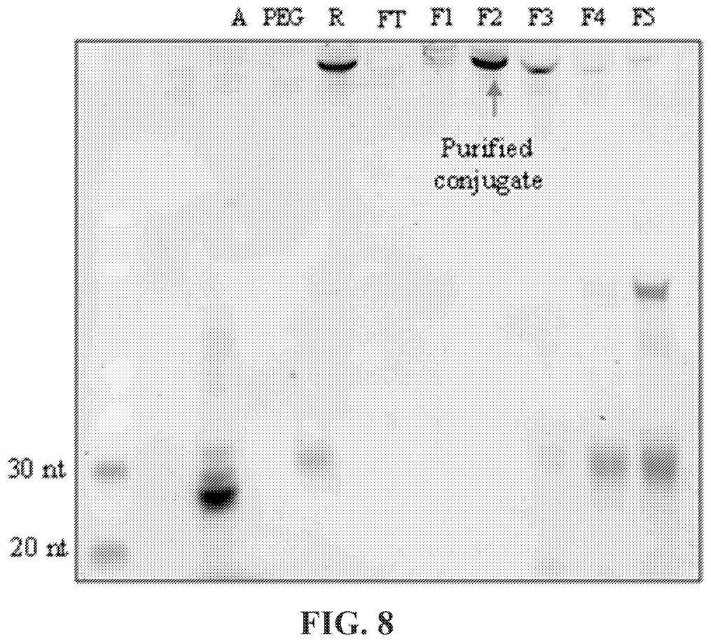

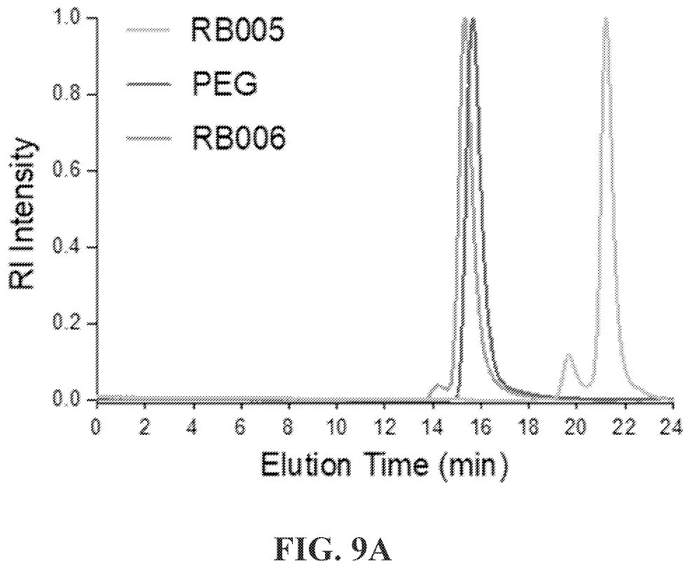

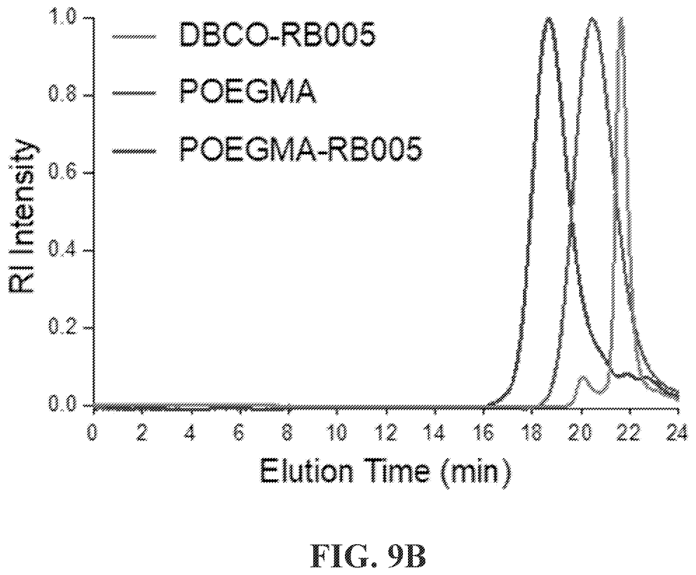

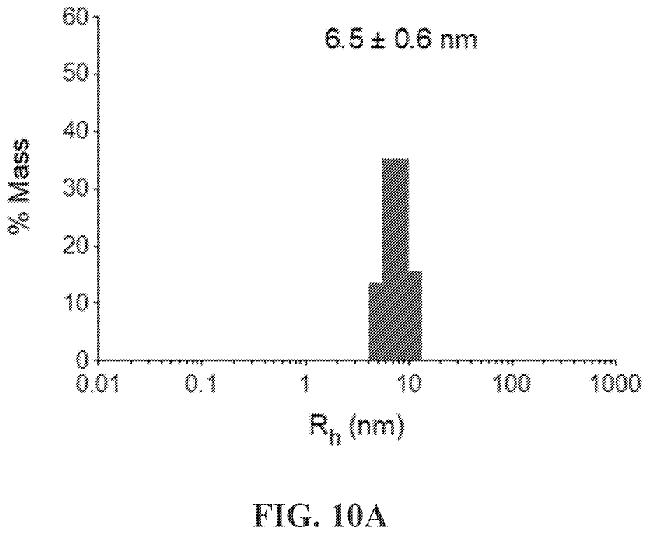

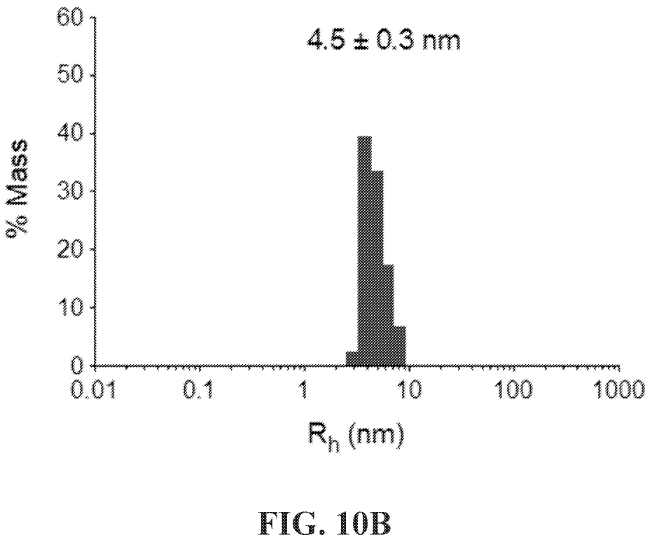

The resulting conjugates were purified by anion-exchange chromatography and were characterized by gel electrophoresis, SEC-MALS, and dynamic light scattering (DLS). TBE-urea polyacrylamide gel electrophoresis analysis of the purification of RB006 by anion-exchange electrophoresis showed the absence of unmodified aptamer in the resulting conjugate (FIG. 8). SEC-MALS showed that the conjugates were monodisperse with a M_{w} of ˜50 kDa and D of <1.1 (FIG. 2B; Table 1; FIG. 9). The PEG and POEGMA conjugates had a significantly larger hydrodynamic size (Rₕ) than RB005, as measured by DLS (Table 1; FIG. 10). POEGMA-RB005 was significantly smaller than RB006 due to its more compact hyperbranched structure than the linear PEG conjugate. Both conjugates had a Rₕ above the renal excretion threshold, defined as the size of serum albumin (˜3.6 nm), which should endow them with an improved PK relative to the parent aptamer. It was also previously reported that stoichiometric PEG and POEGMA conjugates with a M_{w} greater than 30 kDa and 25 kDa, respectively, showed prolonged circulation. Together, these data indicated that the POEGMA and PEG conjugates were site-specific and stoichiometric (1:1) and had comparable physical properties.

TABLE 1
Characterization of RB005 variants and intermediates used
in the synthesis. The Mₙ, M_{w}, and Ð were determined
from the SEC-MALS data given in FIG. 2B (n = 3). Rₕ was
determined from the DLS data in FIG. 10 (n = 10).
Data were reported as mean ± standard error of the mean (SEM).

| Compound | Mₙ [kDa] | M_{w} [kDa] | Ð | Rₕ [nm] |
|---|---|---|---|---|
| RB005 | 10.2 | 10.3 | 1.01 | 1.7 ± 0.3 |
| DBCO-RB005 | 10.6 | 10.7 | 1.01 | 2.2 ± 0.5 |
| POEGMA | 36.1 | 38.3 | 1.06 | 4.1 ± 0.4 |
| POEGMA- | 45.3 | 47.6 | 1.05 | 4.5 ± 0.3 |
| RB005 | | | | |
| PEG | 38.1 | 38.9 | 1.02 | 6.3 ± 0.5 |
| RB006 | 52.0 | 52.5 | 1.01 | 6.5 ± 0.6 |

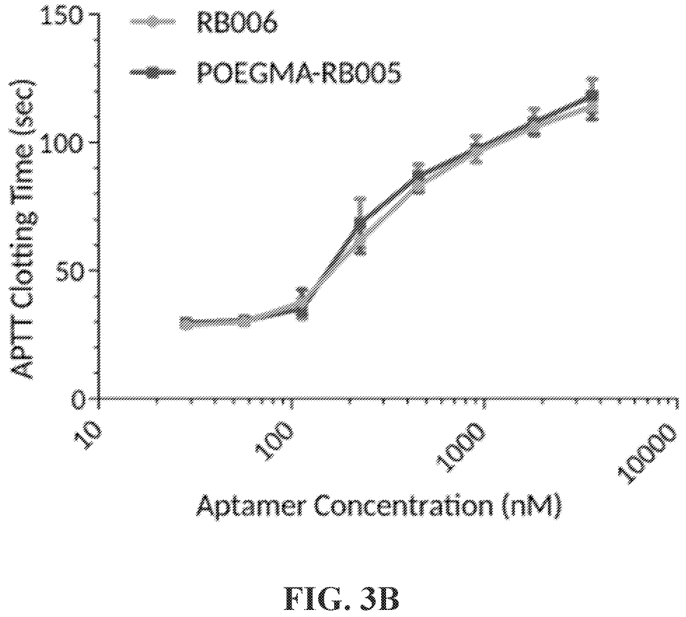

Reversible in vitro anticoagulant activity We next investigated if POEGMA-RB005 could inhibit FIXa activity in human plasma. FIXa complexed with FVIIIa induces fibrin clot formation by activating FX on the platelet surface, leading to blood coagulation. RB005 variants exert antithrombotic activity by inhibiting FIXa cleavage of FX, resulting in prolonged coagulation time (FIG. 3A). We used an activated partial thromboplastin time (aPTT) assay, which is universally used in the clinic to measure the coagulation time. Pooled normal human plasma clotted in ˜30 seconds in the absence of treatment (FIG. 3B). The conjugates extended the plasma clotting time up to ˜4-fold in a concentration-dependent manner. POEGMA-RB005 showed in vitro antithrombotic activity comparable to RB006 (p=0.92).

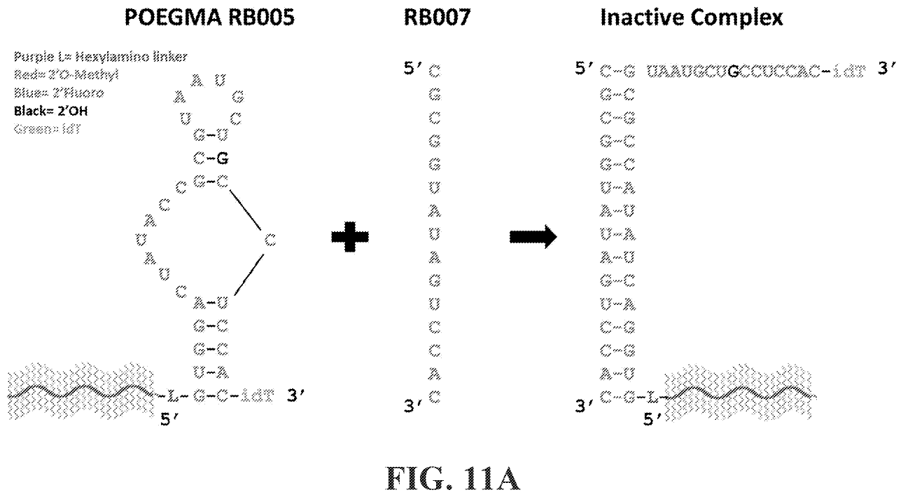

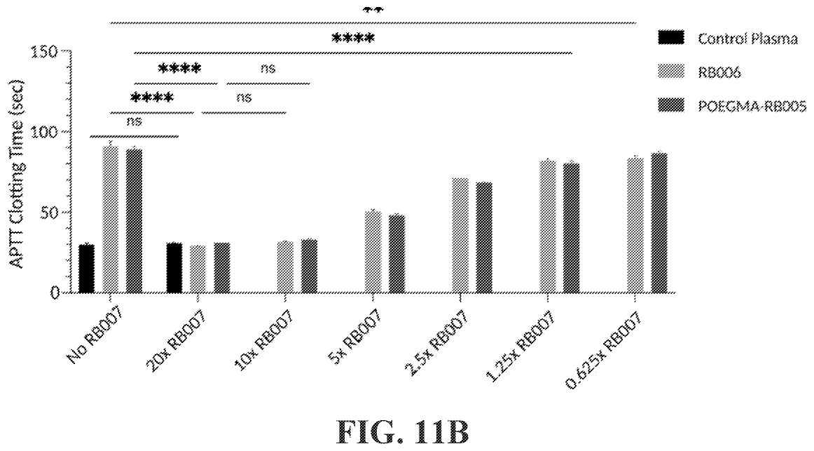

POEGMA-RB005 was also tested for the reversal of its anticoagulant activity using RB007 as an antidote. RB007 is an RNA oligonucleotide that is partially complementary to RB005. It blocks RB005 variants' binding to FIXa by altering their secondary structure via Watson-Crick base pairing, resulting in the reversal of anticoagulant activity (FIG. 11A). RB007 alone did not affect clotting (FIG. 11B). In addition, RB007 inhibited the PEG and POEGMA conjugates' anticoagulant activity in a dose-dependent manner, indicating that the action of POEGMA-RB005 is reversible. Together, these data indicate that POEGMA-RB005 successfully binds to FIXa, inhibits FIXa activity, and shows reversible antithrombotic activity comparable to RB006.

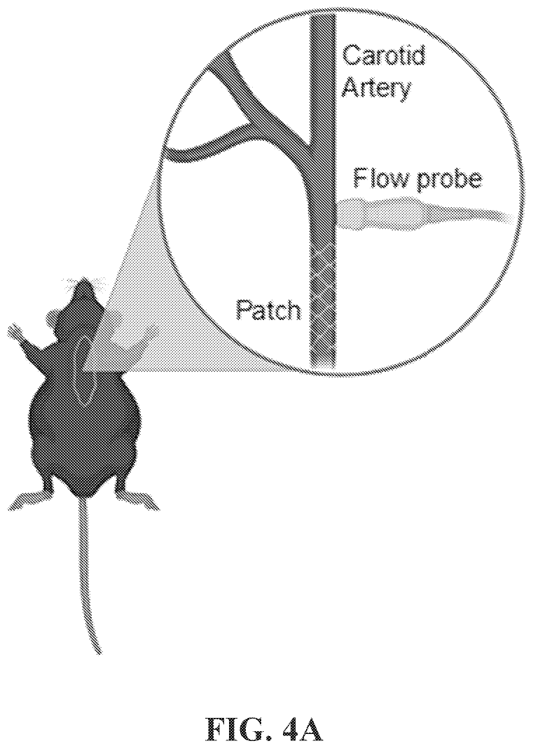

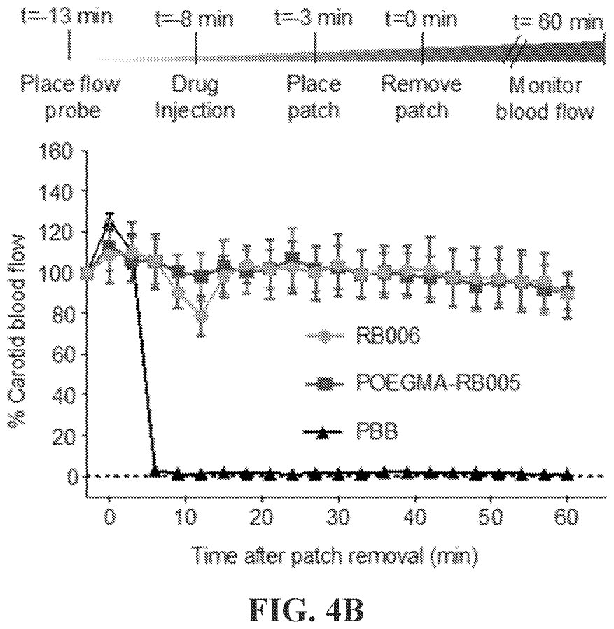

In Vivo Efficacy: We next investigated if POEGMA-RB005 has in vivo antithrombotic efficacy comparable to RB006. We used a ferric chloride (FeCl₃)-induced murine thrombosis model, where FeCl₃ is used to create a vascular injury leading to occlusive thrombosis in arteries. Briefly, a distal flow probe was placed on the carotid artery of mice to measure the blood flow, followed by intravenous (i.v.) drug administration. Next, a FeCl₃-saturated patch was applied to the carotid artery to induce endothelial damage and trigger thrombosis, followed by removal of the patch and monitoring the blood flow (FIG. 4A) for 60 min. The absence of a significant reduction in blood flow after the patch was removed would indicate that the drug prevented the formation of an occlusive thrombus.

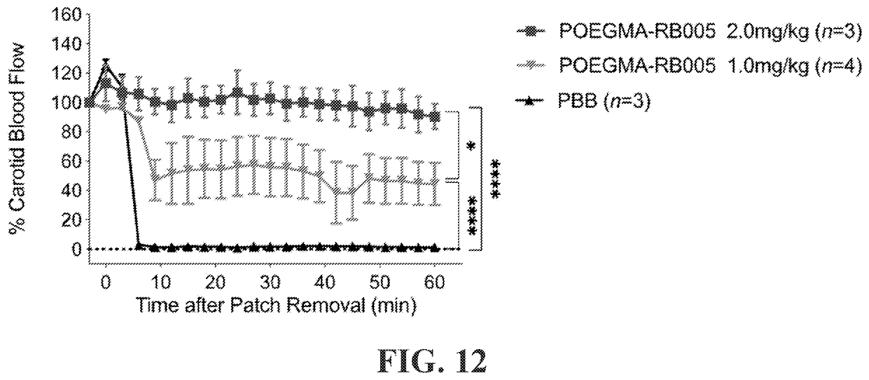

We first determined an optimal injection dose of POEGMA-RB005 in a dose-escalation study in the murine thrombosis model (FIG. 12). POEGMA-RB005 showed a dose-dependent efficacy. An i.v. injection dose of 1 mg kg⁻¹ resulted in only a moderate antithrombotic response, indicated by the partially blocked blood flow, whereas POEGMA-RB005 injected at 2 mg kg⁻¹ dose prevented thrombosis, indicated by the lack of occlusive thrombi. The injection dose of 2 mg kg⁻¹ was hence chosen as the dose and kept constant across all in vivo studies.

We next tested the in vivo efficacy of POEGMA-RB005 by i.v. administration in mice at an injection dose of 2 mg kg⁻¹ using an equivalent dose of RB006 as a positive control and an equivalent injection volume of platelet-binding buffer (PBB) as a negative control. The blood flow occluded within ˜10 minutes of PBB administration due to the formation of occlusive thrombi in the carotid artery (FIG. 4B). In contrast, POEGMA-RB005 treated mice did not show a significant change in the blood flow throughout the experiment, indicating that POEGMA-RB005 successfully maintained the carotid artery's patency. No difference was observed in the antithrombotic efficacy between RB006 and POEGMA-RB005, possibly due to their similar FIXa binding and in vitro antithrombotic activities.

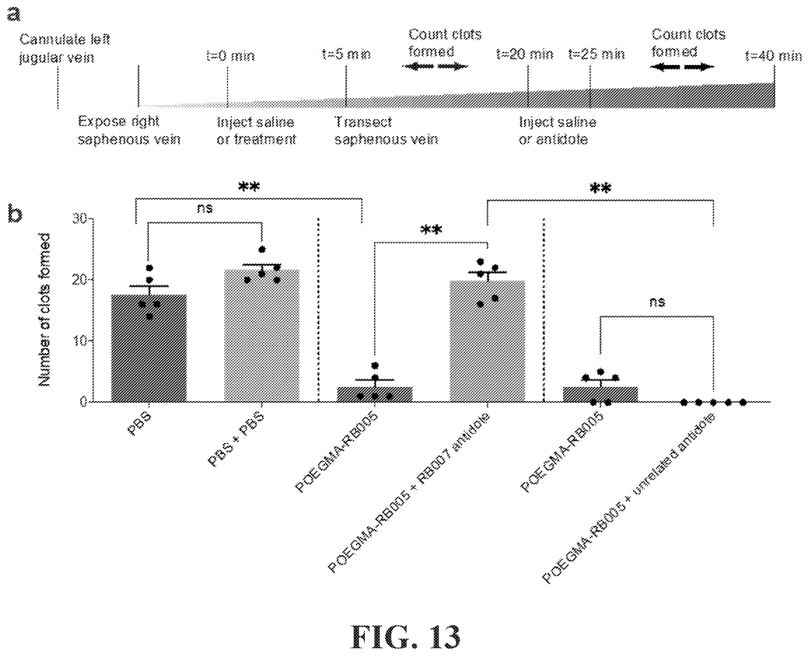

We also tested the reversibility of POEGMA-RB005's in vivo anticoagulant activity. The ability of an antidote oligonucleotide (RB007) to reverse the anticoagulant ability of POEGMA-RB005 was evaluated in a murine saphenous vein bleeding model (FIG. 13A). In this experiment, the saphenous vein is exposed and transected by a 23-gauge needle, and the number of times the damaged vessel stops bleeding over 15 minutes is assessed by disrupting clots as soon as they form. Fewer disruptions correlate with reduced clotting and increased bleeding. The vehicle group stopped bleeding 18±2 times over the initial 15 minutes (n=5), and then 22±1 times in the subsequent 15-minute observation window (FIG. 13B). In contrast, animals receiving POEGMA-RB005 at a dose of 2.0 mg kg⁻¹ stopped bleeding 3±1 times initially. Adding the specific antidote (RB007) at a 10:1 molar excess and measuring clot formation again resulted in restoration of normal hemostasis, with 20±1 clots forming in 15 minutes, which is similar to animals that never received the aptamer (n=5). The POEGMA-RB005 group given an unrelated control antidote, at a 10:1 molar excess, continued to bleed (n=5). Therefore, we concluded that a specific antidote oligonucleotide (RB007) can rapidly restore hemostasis by reversing the anticoagulant aptamer POEGMA-RB005's anticoagulant activity within 5 minutes in vivo.

PEG Antigenicity: Having demonstrated that POEGMA-RB005 retained the favorable in vivo anticoagulant efficacy of RB006, we next investigated if it eliminated RB006's PEG antigenicity. We first tested the reactivity of POEGMA-RB005 towards PEG antibodies of varied origins using indirect enzyme-linked immunosorbent assay (ELISA). Competitive ELISA was used to confirm the results because it eliminates the significant drawbacks of indirect ELISA, where antigen recognition occurs on a solid surface, resulting in differences in the amounts of antigen adsorbed onto the surface that can skew the results.

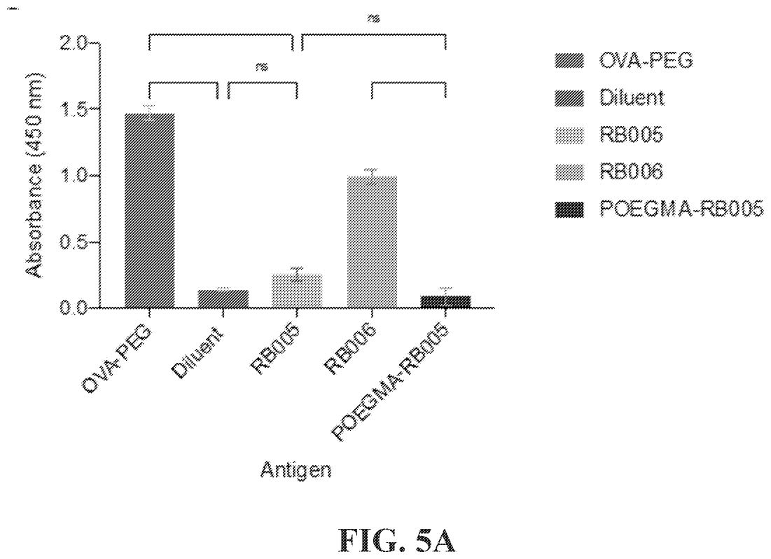

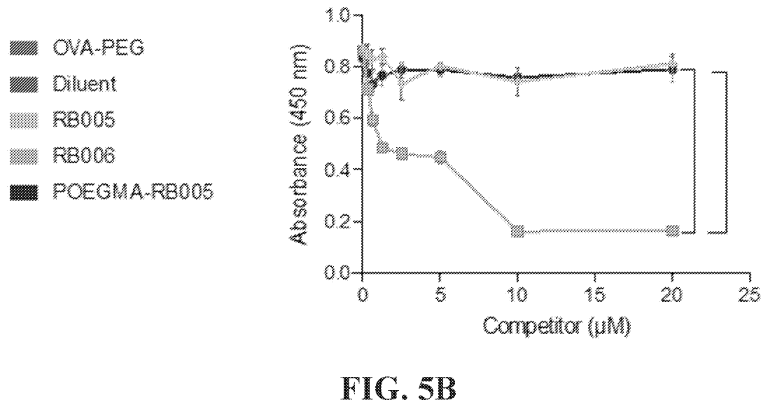

We first tested if POEGMA-RB005 showed reactivity to induced polyclonal PEG antibodies in an indirect ELISA. We used a murine plasma sample available from an earlier study in which mice were repeatedly immunized with an emulsion of PEGylated ovalbumin (OVA) in Freund's adjuvant to induce a high titer (˜120 μg/ml) of strictly Immunoglobulin (Ig) M-class, polyclonal PEG-specific antibody response. In indirect ELISA, OVA-PEG (positive control) showed binding to antibodies present in the plasma sample (FIG. 5A), indicated by the higher absorbance than diluent. Similarly, RB006 bound to the PEG antibodies present in the plasma sample. RB005, which shares the same nucleotide sequence with RB006 but lacks PEG, showed no reactivity to the PEG antibodies, indicating that the antibody binding was PEG-specific. Notably, POEGMA-RB005 showed no reactivity at all towards the PEG antibodies. The lack of reactivity was likely because the OEG side-chain length was shorter than the PEG antibody epitope.

We next confirmed these findings by a competitive ELISA. To carry out a competitive ELISA, a dilution series of RB006, and RB005-POEGMA (n=4) or RB005 (PEG-minus negative control) were added to a murine plasma sample that contains polyclonal PEG antibodies. This mixture was incubated on exendin-PEG conjugate coated microwell-plates. RB006 competed with the adsorbed exendin-PEG for binding with the PEG antibodies present in the murine plasma sample (FIG. 5B), indicated by the decreasing absorbance at increasing concentrations of RB006. RB005 and POEGMA-RB005 conjugate signals remained unchanged as a function of their concentration, suggesting that they did not compete with the adsorbed exendin-PEG for binding to the PEG antibodies in the murine plasma sample.

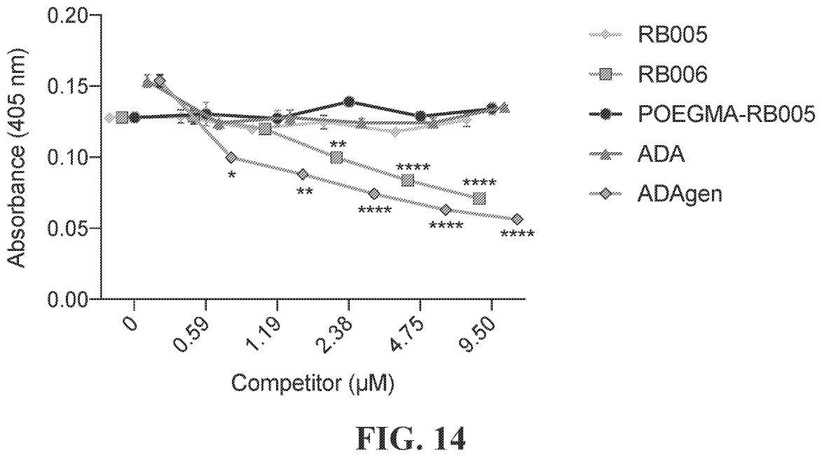

Finally, we tested the reactivity of POEGMA-RB005 to pre-existing human PEG antibodies present in patient plasma samples with pre-existing PEG antibodies. We used adenosine deaminase (ADA)—a protein drug—and its FDA-approved PEGylated formulation (ADAgen) to validate our results. RB006 and ADAgen competed with the patient-derived PEG antibodies, while ADA and RB005 showed no binding due to the lack of PEG moiety (FIG. 14). Notably, POEGMA-RB005 did not compete with the patient-derived PEG antibodies, corroborating with our previous results. Together, these studies indicated that POEGMA-RB005 showed no reactivity towards murine and human PEG antibodies.

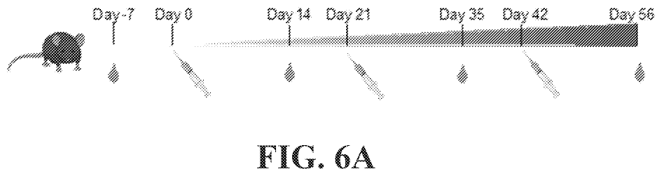

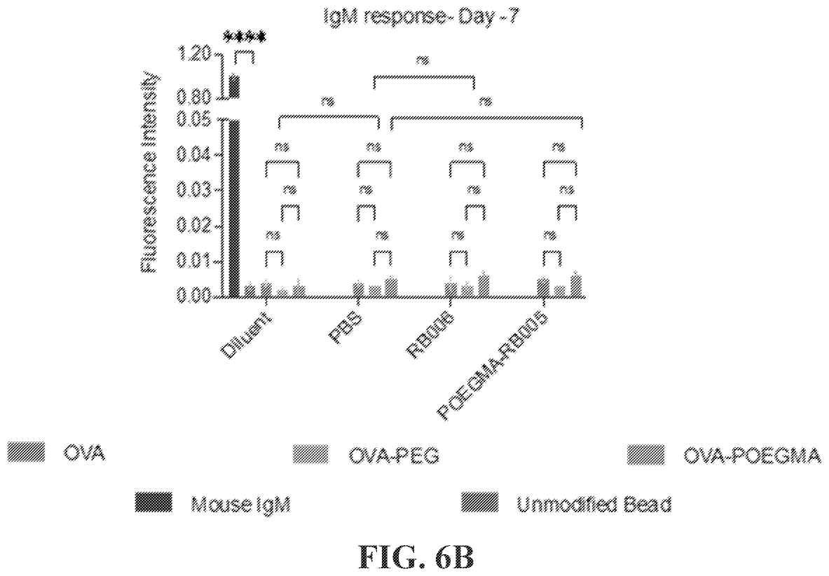

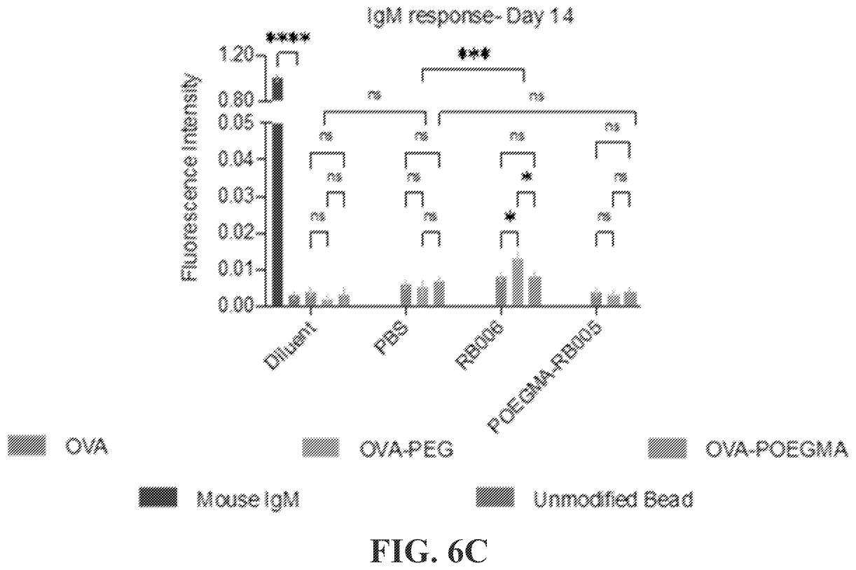

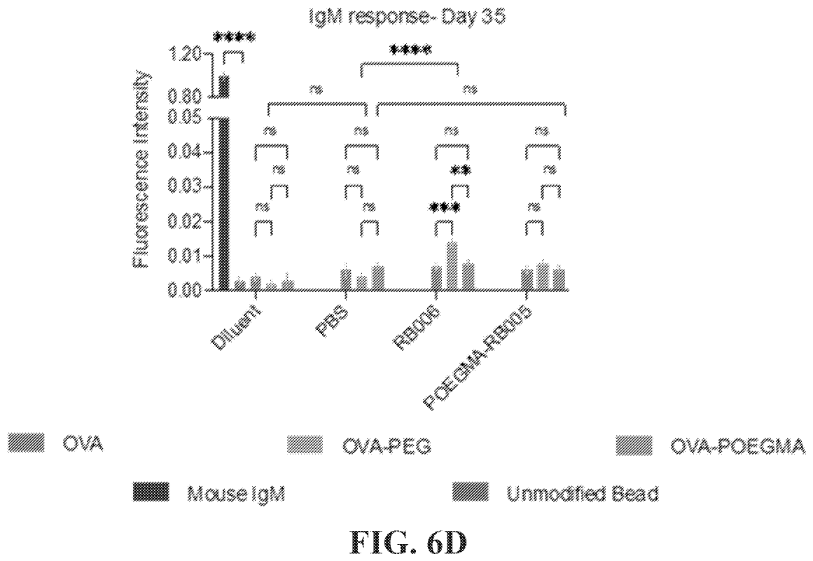

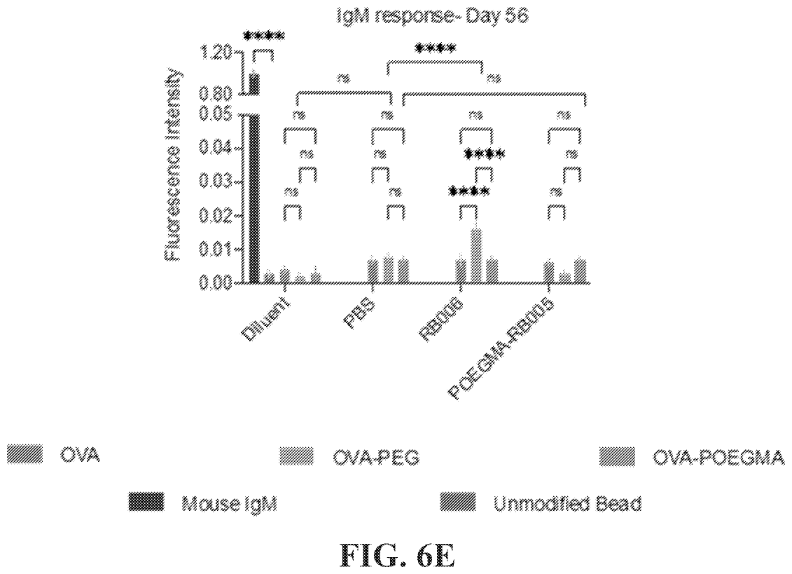

Immunogenicity: Having demonstrated that POEGMA-RB005 eliminated the PEG antigenicity of RB006 measured in vitro, we next investigated the immunogenicity of POEGMA in mice, defined as the induction of anti-POEGMA antibodies upon treatment. We repeatedly administered endotoxin-free sterilized PBS (negative control), RB006, and POEGMA-RB005 into naïve C57BL/6J (n=10) mice at a 2 mg kg⁻¹ dose, followed by collecting blood samples (see the dosing and sampling regimen in FIG. 6A). We chose the subcutaneous (s.c.) injection route because it better reveals the immunogenicity of large-molecule therapeutics by exposing them to the immune system during lymphatic absorption from the s.c. space into the blood. The blood samples were processed into sera, followed by an assessment of induced anti-drug antibodies (ADA).

We used a Luminex multiplexed assay validated in an earlier study, with minor modifications to assess the specificity (anti-PEG or anti-POEGMA) and subtype (IgG and IgM) of the ADA response. Briefly, stoichiometric (1:1) OVA conjugates of PEG and POEGMA were covalently coupled onto separate Luminex magnetic bead sets to capture PEG- and POEGMA-specific antibodies, respectively. The OVA-coupled bead set was used as a control for cross-reactivity to ensure that the captured antibodies were not specific to OVA, due to accidental prior exposure of the mice to the OVA antigen. The fluorescent barcoding of the Luminex beads allowed us to multiplex the drug-coupled bead sets and simultaneously measure the signal detected by each of them in any given serum sample. We used mouse IgG- or IgM-coupled beads incubated in the PEG-free assay diluent, which is 0.2% (w/v) T-Block protein-based blocking reagent (Thermo Scientific) in PBS, as positive controls for the assay. A mixture of OVA, OVA-PEG, and OVA-POEGMA coupled beads incubated in the assay diluent was used as a negative control.

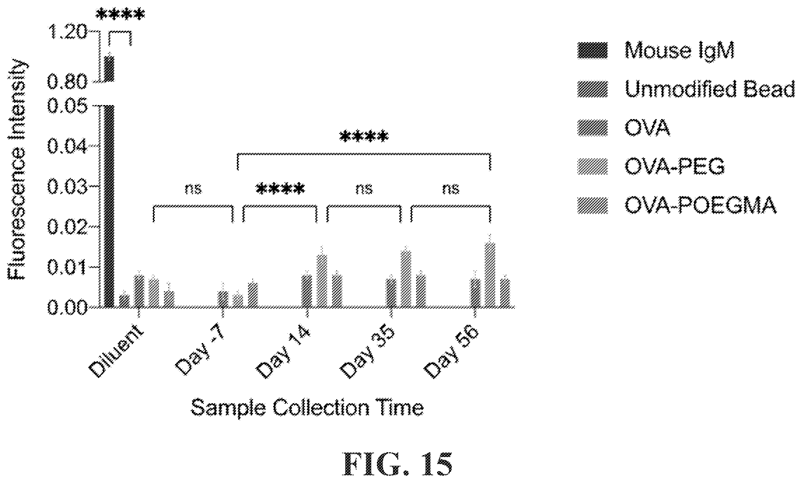

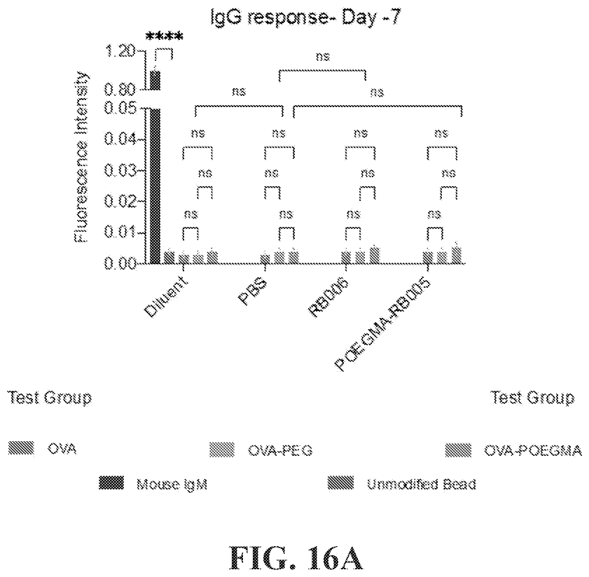

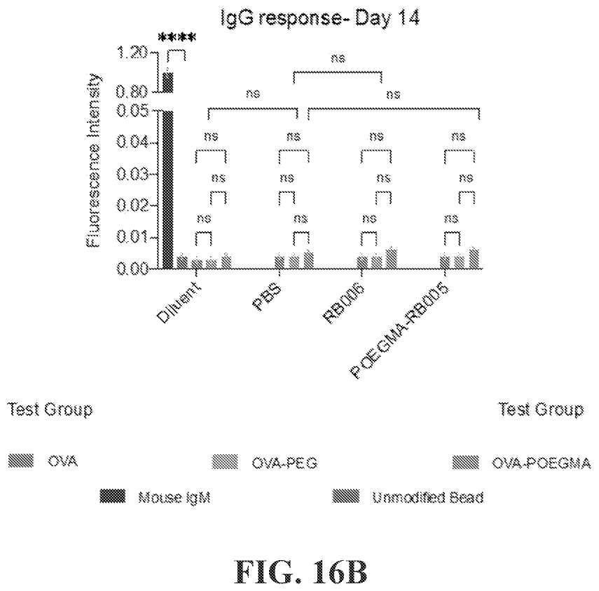

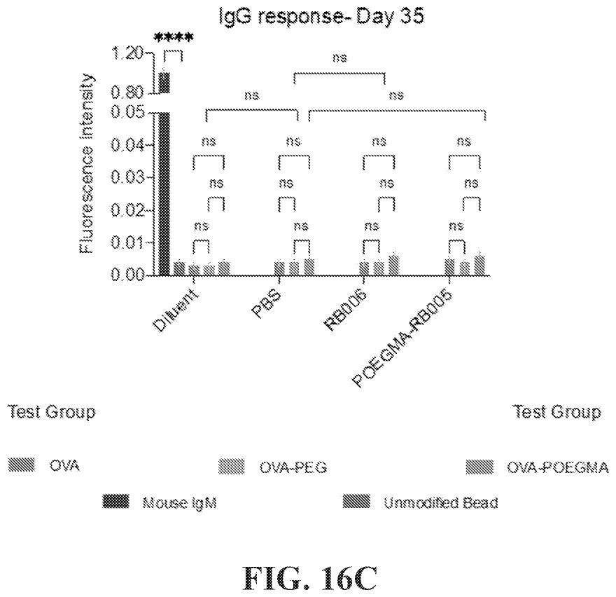

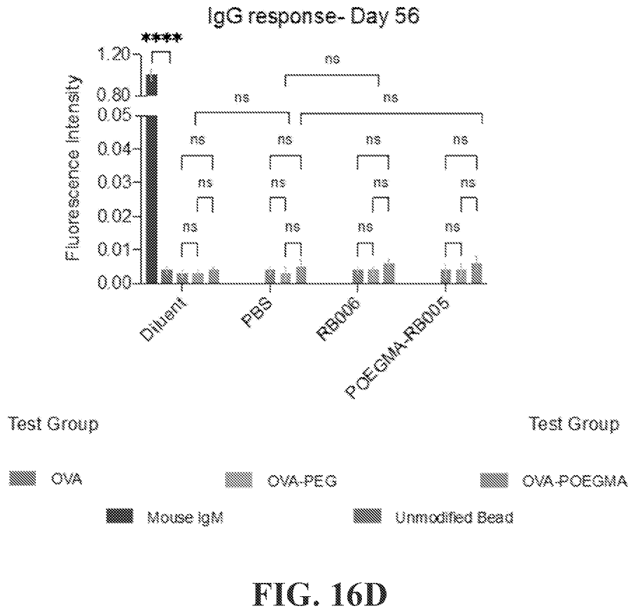

The mouse IgM-coupled bead had a significant fluorescence signal, while OVA, OVA-PEG, and OVA-POEGMA coupled beads incubated in the PEG-free diluent had only a minimal response, indicating that the Luminex assay has a minimal background (FIG. 6B, FIG. 6C, FIG. 6D, and FIG. 6E). All samples tested pre-dose (FIG. 6B) and all post-dose PBS-treated (FIG. 6C, FIG. 6D, and FIG. 6E) mice sera did not have any anti-OVA, anti-PEG, or anti-POEGMA antibodies, as expected. These samples also established the baseline sensitivity of the assay. Notably, RB006 induced a significant ADA response in mice to PEG 14 days after the first injection, indicated by the magnitude of the fluorescence signal detected from the OVA-PEG bead but not from the OVA bead (FIG. 6C). This IgM response of RB006-treated mice strengthened modestly with repeated injection of RB006, as seen by the increase in the magnitude of the fluorescence signal for sera tested at days 35 and 56. The OVA-coupled bead incubated in serum did not differ from the diluent, indicating the lack of cross-reactivity towards OVA (FIG. 6B, FIG. 6C, FIG. 6D, and FIG. 6E). These results suggested that the induced ADA response for RB006 was PEG-specific. We further analyzed the data for RB006 IgM titers. A test for statistical significance of RB006 exposure after repeated antigen injections showed that although the PEG-specific ADA response persisted throughout the ˜2-month duration of the experiment, there was no significant increase in the IgM titer with increasing number of injections over the course of 56 days (FIG. 15). The low IgM titer was attributed to the minimal immunogenicity of chemically modified RNA aptamers, as the PEG specific antibody titer of PEG conjugates has been found to be closely correlated with the immunogenicity of the conjugation partner. No maturation into an IgG-class ADA response was observed for RB006 (FIG. 16). Strikingly, POEGMA-RB005 did not induce an IgM (FIG. 6B, FIG. 6C, FIG. 6D, and FIG. 6E) or IgG (FIG. 16) anti-POEGMA immune response, indicated by the lack of a statistically significant signal detected from the OVA-POEGMA beads compared to OVA-POEGMA coupled beads exposed to diluent or PBS. Together, these results indicate that POEGMA-RB005 eliminates the immunogenicity limitation of the PEGylated conjugate in mice.

### DISCUSSION

Thrombosis is the primary cause of death worldwide. Despite the advances in developing safer antithrombotic agents, they are limited with the significant risk of bleeding. The bleeding risk is even higher for patients undergoing highly prothrombotic clinical procedures, including percutaneous coronary intervention (PCI, termed angioplasty), coronary artery bypass graft (CABG) surgery, and dialysis. Significant toxicity and drug-induced bleeding associated with the FDA-approved parenteral ROAs, such as unfractionated heparin, lepirudin, bivalirudin, and argatroban, have prompted efforts to identify reversible ROAs that eliminate these limitations. Before its Phase 3 clinical trial was halted, RBOO6, —a PEG conjugate of a FIX binding aptamer—together with the complementary antidote sequence capable of titrating and rapidly reversing the anticoagulant activity of RB006, was evaluated in >2,000 patients in Phase 1 and Phase 2 clinical trials. Phase 2 studies suggested that the aptamer-antidote pair significantly reduced ischemic events and limited bleeding in PCI patients compared to heparin. Unfortunately, the development of this drug was derailed by the rare events of PEG antigenicity, when a small fraction (<1%) of patients developed life threatening anaphylactoid reactions with one fatality amongst the 1616 patients receiving RB006 in the Phase 3 clinical trials.

In response to the failure of RB006 in clinical trials, we decided to replace PEG by POEGMA to investigate if it would eliminate the PEG antigenicity and immunogenicity issues of RB006 in mice, and thereby provide a way to rescue a PEGylated aptamer drug that failed in late-stage clinical development due to these PEG issues. We hence conjugated the parent ROA aptamer—RB005—with POEGMA, yielding a well-defined POEGMA-RB005 conjugate that is—similar to RB006—site-specific, with the polymer conjugated to 5′ end of the aptamer and has a 1:1 stoichiometry of polymer: aptamer. This next generation PEG-like conjugate is similar to RB006 in its physical properties, with a similar Rₕ that is slightly greater than the renal excretion threshold, which should endow it—like the PEGylated aptamer—with prolonged blood circulation. The POEGMA and PEG conjugates exhibited similar in vitro FIXa binding and potent antithrombotic activity that is rapidly reversible by a complementary RNA antidote (RB007). The rapid reversal of activity in ˜5 minutes in a dose-dependent manner is important, as it allows for precise temporal control over blood coagulation. In a murine model of thrombosis, the conjugates prevented the formation of occlusive thrombi and maintained the patency of the carotid artery, indicating that POEGMA formulated aptamer preserved RB006's pharmacodynamic (PD) benefits in mice.

Our results show that POEGMA-RB005 eliminates the PEG antigenicity problem-defined as the binding of a PEGylated drug by pre-existing PEG antibodies in human patients—that ultimately led to the early termination of RB006 Phase 3 clinical trial, where the harmful interactions of pre-existing PEG antibodies with RB006 resulted in severe allergic reactions and anaphylaxis. The POEGMA formulated aptamer showed no reactivity towards PEG antibodies of varied origin as measured in vitro, suggesting that it could potentially address the growing problem of PEG antigenicity and avoid causing severe side-effects. The lack of PEG antigenicity can be attributed to POEGMA's hyperbranched architecture that disrupts the PEG antibodies' binding epitopes by stacking EG3 side chains on a backbone. These results indicate that, unlike RBOO6, POEGMA-RB005 has the potential to be safely used in PEG antibody-positive population. As illustrated by the halted clinical trial of RBOO6, and the withdrawal of several PEGylated drugs from the market, the effect of pre-existing PEG antibodies may be a growing concern due to their prevalence in up to 70% of the human population at varying titers who are naïve to PEGylated therapeutics. As illustrated with the rare but severe anaphylactoid responses to Pfizer and Moderna vaccines for severe acute respiratory syndrome coronavirus-2 (SARS-CoV-2), where PEG in their liposome formulation is hypothesized to be the culprit, this rare problem of pre-existing PEG antibody reactivity could become more prevalent in future, as billions of individuals have now been exposed to PEG via vaccination with the Moderna and Pfizer SARS-CoV-2 messenger RNA (mRNA) vaccines.

In addition to PEG's antigenicity, the POEGMA formulated aptamer also did not show the PEG immunogenicity limitation of RB006 in mice-defined as the de novo generation of antibodies against the aptamer-polymer conjugate. RB006 induced a persistent IgM-class PEG-specific immune response, while POEGMA-RB005 treatment did not generate anti-POEGMA antibodies. These results are consistent with the literature, where PEGylated drugs induce PEG antibodies upon treatment, likely because PEG's repetitive structure crosslinks the B-cell receptors (BCR) and result in a T-cell independent B cell immune response. This type of immune response is characterized by the release of IgM-class PEG antibodies, although differences in the Ig class are seen with varied conjugation partners and host species. We attributed POEGMA's lack of immunogenicity in mice to its hyperbranched structure presenting much shorter EG3 units as side chains that may be able to prevent the crosslinking of the BCRs. These results suggest that POEGMA-RB005 has the potential not to pose a safety risk upon repeated drug injections.

It is noted that this is the first conjugate of POEGMA coupled to an RNA or DNA aptamer. Although we focused on a FIX aptamer herein, this technology applies broadly to all RNA and DNA aptamers and could help resuscitate the development of PEGylated RNA aptamers as therapeutics. Only one PEGylated aptamer—Pegaptanib (Macugen)—has successfully reached the market because it is not affected by the harmful effects of the pre-existing PEG antibodies owing to its local administration into the eye—an immune-privileged organ—and low dose, limiting the strength of the response that would ensue. Given that 6 out of the 7 RNA aptamers currently in the clinical trials are PEGylated, the disclosed PEG-like conjugate technology has the potential to combat the PEG antigenicity problem limiting the use of PEGylated RNA aptamers.

In conclusion, we have synthesized the first POEGMA conjugate of an RNA aptamer drug. The resulting POEGMA conjugate eliminated the PEG antigenicity measured in vitro and immunogenicity problems of a PEGylated drug in mice while preserving its benefits, suggesting that it may have the translational potential to meet the clinical need for a rapidly reversible ROA in humans.

It is understood that the foregoing detailed description and accompanying examples are merely illustrative and are not to be taken as limitations upon the scope of the invention.

For reasons of completeness, various aspects of the invention are set out in the following numbered clauses:

Clause 1. A conjugate comprising: an aptamer; and a poly[oligo(ethylene glycol) methyl ether methacrylate] (POEGMA) conjugated to the aptamer, wherein the POEGMA has a poly(methyl methacrylate) backbone and a plurality of side chains covalently attached to the backbone, each side chain comprising 1 to 6 monomers of ethylene glycol (EG) repeated in tandem, wherein the conjugate does not induce an anti-POEGMA antibody response.

Clause 2. The conjugate of clause 1, wherein the aptamer comprises about 15 nucleotides to about 100 nucleotides.

Clause 3. The conjugate of clause 1 or clause 2, wherein the aptamer comprises at least one stem and at least one loop.

Clause 4. The conjugate of any one of clauses 1-3, wherein the aptamer is capable of binding a blood protein.

Clause 5. The conjugate of any one of clauses 1-4, wherein the aptamer comprises modified nucleotides.

Clause 6. The conjugate of any one of clauses 1-5, wherein the aptamer comprises SEQ ID NO: 1.

Clause 7. The conjugate of any one of clauses 1-6, wherein the conjugate is not reactive with pre-existing anti-PEG antibodies in a subject.

Clause 8. The conjugate of any one of clauses 1-7, wherein the POEGMA has a number average molecular weight of about 5 kDa to about 50 kDa.

Clause 9. The conjugate of any one of clauses 1-8, wherein each side chain comprises 3 monomers of EG repeated in tandem.

Clause 10. The conjugate of any one of clauses 1-9, wherein the POEGMA is conjugated to a 5′ end of the aptamer.

Clause 11. A method of making a polymer-aptamer conjugate, the method comprising: conjugating a poly[oligo(ethylene glycol) methyl ether methacrylate] (POEGMA) having a poly(methyl methacrylate) backbone and a plurality of side chains covalently attached to the backbone, each side chain comprising 1 to 6 monomers of ethylene glycol (EG) repeated in tandem to a 5′ end of an aptamer to provide a conjugate, wherein the conjugate does not induce an anti-POEGMA antibody response.

Clause 12. The method of clause 11, wherein the POEGMA is functionalized with an azide group and the aptamer is functionalized with a dibenzocyclooctyne group prior to conjugating.

Clause 13. The method of clause 11 or clause 12, wherein the aptamer comprises about 15 nucleotides to about 100 nucleotides.

Clause 14. The method of any one of clauses 11-13, wherein the aptamer comprises SEQ ID NO: 1.

Clause 15. The method of any one of clauses 11-14, wherein the POEGMA has a number average molecular weight of about 5 kDa to about 50 kDa.

Clause 16. The method of any one of clauses 11-15, wherein each side chain comprises 3 monomers of EG repeated in tandem.

Clause 17. A method of controlling coagulation in a subject, the method comprising administering to a subject in need thereof a therapeutically effective amount of the conjugate of any one of clauses 1-10, wherein administering the conjugate prevents or reduces blood clot formation in the subject.

Clause 18. The method of clause 17, further comprising administering to the subject an antidote oligonucleotide in a therapeutically effective amount to neutralize the conjugate.

Clause 19. The method of clause 17 or clause 18, wherein the antidote oligonucleotide comprises SEQ ID NO: 2.

Clause 20. The method of any one of clauses 17-19, wherein the subject suffers from FV Leiden or atrial fibrillation, or is at risk of having deep vein thrombosis, a stroke, a heart attack, or a pulmonary embolism.

Clause 21. The method of any one of clauses 17-20, wherein the subject is a surgery patient.

### SEQUENCES

SEQ ID NO: 1: RB005 (C6L) mGmUmGmGmAfCfUmAfUmAfCfCmGfCmGfUmAmAfUmGfCmUrGmCfCfUmCmCmAm CidT-3′ mC, mG, mU, and mA represent 2′O-methyl Cytosine, 2′O-methyl Guanosine, 2′O-methyl Uridine, and 2′O-methyl Adenosine, respectively; fC and fU represent 2′Fluoro Cytosine and 2′Fluoro Uridine; rG is 2′Hydroxy Guanosine; idT is inverted deoxy thymidine; C6L is a hexylamino linker
SEQ ID NO: 2: RB007 mCmGmCmGmGmUmAmUmAmGmUmCmCmAmC, where mC, mG, mU, and mA represent 2′O-methyl Cytosine, 2′O-methyl Guanosine, 2′O-methyl Uridine, and 2′O-methyl Adenosine, respectively.

What is claimed is:

1. A conjugate comprising:
an aptamer; and
a poly[oligo(ethylene glycol) methyl ether methacrylate] (POEGMA) conjugated to the aptamer, wherein the POEGMA has a poly(methyl methacrylate) backbone and a plurality of side chains covalently attached to the backbone, each side chain comprising 1 to 6 monomers of ethylene glycol (EG) repeated in tandem,
wherein the conjugate does not induce an anti-POEGMA antibody response.

2. The conjugate of claim 1, wherein the aptamer comprises about 15 nucleotides to about 100 nucleotides.

3. The conjugate of claim 1, wherein the aptamer comprises at least one stem and at least one loop.

4. The conjugate of claim 1, wherein the aptamer is capable of binding a blood protein.

5. The conjugate of claim 1, wherein the aptamer comprises modified nucleotides.

6. The conjugate of claim 1, wherein the aptamer comprises SEQ ID NO: 1.

7. The conjugate of claim 6, wherein the conjugate is not reactive with pre-existing anti-PEG antibodies in a subject.

8. The conjugate of claim 1, wherein the POEGMA has a number average molecular weight of about 5 kDa to about 50 kDa.

9. The conjugate of claim 1, wherein each side chain comprises 3 monomers of EG repeated in tandem.

10. The conjugate of claim 1, wherein the POEGMA is conjugated to a 5′ end of the aptamer.

11. A method of making a polymer-aptamer conjugate, the method comprising:
conjugating a poly[oligo(ethylene glycol) methyl ether methacrylate] (POEGMA) having a poly(methyl methacrylate) backbone and a plurality of side chains covalently attached to the backbone, each side chain comprising 1 to 6 monomers of ethylene glycol (EG) repeated in tandem to a 5′ end of an aptamer to provide a conjugate,
wherein the conjugate does not induce an anti-POEGMA antibody response.

12. The method of claim 11, wherein the POEGMA is functionalized with an azide group and the aptamer is functionalized with a dibenzocyclooctyne group prior to conjugating.

13. The method of claim 11, wherein the aptamer comprises about 15 nucleotides to about 100 nucleotides.

14. The method of claim 11, wherein the aptamer comprises SEQ ID NO: 1.

15. The method of claim 11, wherein the POEGMA has a number average molecular weight of about 5 kDa to about 50 kDa.

16. The method of claim 11, wherein each side chain comprises 3 monomers of EG repeated in tandem.

17. A method of controlling coagulation in a subject, the method comprising administering to a subject in need thereof a therapeutically effective amount of the conjugate of claim 1, wherein administering the conjugate prevents or reduces blood clot formation in the subject.

18. The method of claim 17, further comprising administering to the subject an antidote oligonucleotide in a therapeutically effective amount to neutralize the conjugate.

19. The method of claim 17, wherein the antidote oligonucleotide comprises SEQ ID NO: 2.

20. The method of claim 17, wherein the subject suffers from FV Leiden or atrial fibrillation, or is at risk of having deep vein thrombosis, a stroke, a heart attack, or a pulmonary embolism.

21. The method of claim 17, wherein the subject is a surgery patient.
